(19) United States
(12) Patent Application Publication
(10) Pub. No.: US 2016/0139150 A1
(43) Pub. Date: May 19, 2016

(54) METHODS FOR DIAGNOSING AND ASSESSING NEUROLOGICAL DISEASES

(71) Applicants: Mony de Leon, New York, NY (US); Henry Rusinek, Great Neck, NY (US)

(72) Inventors: Mony de Leon, New York, NY (US); Henry Rusinek, Great Neck, NY (US)

(21) Appl. No.: 14/482,439

(22) Filed: Sep. 10, 2014

Related U.S. Application Data
(60) Provisional application No. 61/877,345, filed on Sep. 13, 2013.

(51) Int. Cl. G01N 33/68

(52) U.S. Cl. CPC G01N 33/6896; G01N 2333/4703; G01N 2333/4709; G01N 2800/2821; G01N 2800/2814

(57) ABSTRACT

The present invention provides methods for diagnosing a neurological disease in a subject, screening for or assessing the risk of developing a neurological disease in a subject, monitoring progression of a neurological disease in a subject, assessing efficacy of a therapy for a neurological disease in a subject, and identifying a subject suffering from a neurological disease that may be successfully treated by an agent that affects levels of a biomarker such as a tau protein or an amyloid beta. The methods generally feature (a) obtaining a cerebrospinal fluid (CSF) sample from a subject; (b) providing a cerebrospinal fluid (CSF) correction factor for CSF obtained from a subject for at least one biomarker; and (c) determining whether the biomarker is present in elevated amounts or concentrations in the cerebrospinal fluid (CSF) sample. The neurological disease may be, for instance, impaired cognition or dementia such as Alzheimer's Disease (AD) or Mild Cognitive Impairment (MCI). The biomarker may be a tau protein such as P-tau₂₃₁ or an amyloid beta (Aβ) such as Aβ₄₂ or Aβ₄₀.

### STATEMENT OF GOVERNMENT RIGHTS

The present invention was developed, at least in part, using government support under NIH-NIA RO1AG12101, R01AG13616, RO1AG022374, and P30AG008051awarded by the National Institutes of Health. Therefore, the Federal Government may have certain rights in the invention.

### FIELD OF THE INVENTION

The present invention provides methods for analyzing biomarkers in cerebrospinal fluid (CSF) using a semi-automated procedure for determining CSF volumes in patients suffering from disease-related increases in CSF volume. The invention further provides methods for diagnosing, assessing risk for developing and monitoring the course of neurological diseases featuring brain pathology including disease characterized by cognitive impairment, such as, for instance, Alzheimer's disease, vascular dementia, boxer's syndrome, multiple sclerosis, amyotrophic lateral sclerosis (ALS), and stroke.

### BACKGROUND OF THE INVENTION

Tau protein is a highly soluble microtubule-associated protein (MAP). In humans, these proteins are mostly found in neurons compared to non-neuronal cells. One of tau's main functions is to modulate the stability of axonal microtubules. Other nervous system MAPs may perform similar functions, as suggested by tau knockout mice, who did not show abnormalities in brain development—possibly because of compensation in tau deficiency by other MAPs. Tau is not present in dendrites and is active primarily in the distal portions of axons where it provides microtubule stabilization but also flexibility as needed. This contrasts with MAP6 (STOP) proteins in the proximal portions of axons which essentially lock down the microtubules and MAP2 that stabilizes microtubules in dendrites. Tau proteins interact with tubulin to stabilize microtubules and promote tubulin assembly into microtubules. Tau has two ways of controlling microtubule stability: isoforms and phosphorylation. Pathologies and dementias of the nervous system such as Alzheimer's disease can result when tau proteins become defective and no longer stabilize microtubules properly.

Six tau isoforms exist in human brain tissue, and they are distinguished by their number of binding domains. Three isoforms have three binding domains and the other three have four binding domains. The binding domains are located in the carboxy-terminus of the protein and are positively-charged (allowing it to bind to the negatively-charged microtubule). The isoforms with four binding domains are better at stabilizing microtubules than those with three binding domains. The isoforms are a result of alternative splicing in exons 2, 3, and 10 of the tau gene. Tau is a phosphoprotein with 79 potential Serine (Ser) and Threonine (Thr) phosphorylation sites on the longest tau isoform. Phosphorylation has been reported on approximately 30 of these sites in normal tau proteins. Phosphorylation of tau is regulated by a host of kinases, including PKN, a serine/threonine kinase. When PKN is activated, it phosphorylates tau, resulting in disruption of microtubule organization. Phosphorylation of tau is also developmentally regulated. For example, fetal tau is more highly phosphorylated in the embryonic CNS than adult tau. The degree of phosphorylation in all six isoforms decreases with age due to the activation of phosphatases. Like kinases, phosphatases too play a role in regulating the phosphorylation of tau. For example, PP2A and PP2B are both present in human brain tissue and have the ability to dephosphorylate Ser396. The binding of these phosphatases to tau affects tau's association with MTs.

Hyperphosphorylation of the tau protein (tau inclusions, pTau) can result in the self-assembly of tangles of paired helical filaments and straight filaments, which are involved in the pathogenesis of Alzheimer's disease and other tauopathies. All of the six tau isoforms are present in an often hyperphosphorylated state in paired helical filaments from Alzheimer's disease (AD) brain. In other neurodegenerative diseases, the deposition of aggregates enriched in certain tau isoforms has been reported. When misfolded, this otherwise very soluble protein can form extremely insoluble aggregates that contribute to a number of neurodegenerative diseases. Recent research suggests that tau may be released extracellularly by an exosome based mechanism in Alzheimer's disease.

Cerebrospinal Fluid (CSF) is a liquid that fills the ventricles of the brain and the spaces between arachnoid matter and pia matter of the brain and spinal cord. As the CSF is in contact with the brain, it has been known for some time that Alzheimer's Disease and other dementia-related illnesses can be diagnosed (and the progression of the illness monitored) by analyzing biomarkers in the CSF. In addition, as more progress is made in the development of drugs that inhibit or slow the progression of Alzheimer's Disease, analyzing the biomarkers can be used to evaluate the effectiveness of a drug on a particular patient, or group of patients. However, the process for analyzing biomarkers in CSF has been slow, cost prohibitive, and has not always provided accurate results. In fact, the sometimes inaccurate results of the prior art can hinder research and development of effective drugs, as well as increase the likelihood that a proper diagnosis of a particular illness may not be made in a timely fashion, or not at all for that matter.

One of the biomarkers of interest has been for some time, the tau protein and related forms thereof. In general, tau proteins are constituents of the neuronal axons. Under conditions of neuronal and synaptic degeneration, as is found with the progression of Alzheimer's Disease, the amount of tau protein in the CSF increases. Surgical research studies for non-Alzheimer related reasons have shown that the ventricular derived CSF concentration of tau (a protein found in all people) is two-fold higher than in the lumbar spinal tap derived CSF which is used for diagnostic purposes.

Lumbar spine derived CSF tau levels are elevated in the early stages of Alzheimer's Disease, perhaps before dementia is even noticeable or detectable in a clinical setting. However, longitudinal studies have reported that both in cases of mild cognitive impairment (MCI), as well as Alzheimer's, tau levels, as well as CSF amyloid beta 1-42 (Aβ₄₂) levels, a marker for the fibrillar amyloid that is deposited in Alzheimer's disease in the form of senile plaques, do not significantly change over time in deteriorating patients.

The surprising result regarding the lack of a significant change in the above-mentioned biomarkers is likely the result of a disease-related increase in the size of the brain's CSF compartment, a compensatory response to the loss of brain tissue. In other words, the amount of CSF increases to compensate for reduced brain volume thus diluting the absolute amount of the biomarker thus causing the afore-mentioned biomarkers in the CSF to remain at similar levels, masking the amount of neuronal deterioration that has taken place when comparing a series of test results taken over time, or compared against predetermined concentration values.

Neuroimaging methods have historically not been used to correct the diluted CSF biomarker. The prior art methods using MRI scans to calculate the CSF compartment heretofore have been cost prohibitive because of the need for highly trained persons to spend sometimes as much as several hours to calculate CSF volume for a single patient by methods that require manual identification and dissection of areas of the brain, with the ventricular size and other CSF containing structures being determined by the number of pixels counted in each of the areas identified. In particular, defining the anatomical boundaries of the ventricular system from MRI scans is complex and time consuming. The MRI scans can number as many as fifty or more “slices” of the ventricle being scanned, thus making the calculations a rather tedious procedure, and subject to error even by highly trained personnel. The prior art has had attempts to provide a semi-automated program to measure the whole brain CSF, but there was absolutely no disclosure, suggestion or motivation that the determination of ventricular size could be used to correct biomarkers in CSF volumes. Accordingly, a more reliable and less expensive way to perform such testing is needed in the art.

CSF tau is increasingly used as a biomarker in the diagnosis of AD and for clinical trial selections (Blennow et al., Lancet, 2006; 368(9533): 387-403; Small et al., Neuron, 2008; 60(4): 534-542). The concentration of Tau has been shown to be related to the magnitude of neuronal degeneration in AD and in normal aging (Blennow et al., Mol Chem Neuropathol, 1995; 26(3): 231-245; Blomberg et al., Dement Geriatr Cogn Disord, 2001; 12(2): 127-132). Further, it is well known that the progressive neurodegeneration of AD is accompanied with evidence for gross brain volume that is readily recognized on MRI and on CT as increased CSF spaces (de Leon et al., AJR Am J Roentgenol, 1989; 152(6): 1257-1262; Frisoni et al., Nat Rev Neurol, 2010; 6(2): 67-77)

While the T-tau levels are believed to be reflective of the tissue damage there has been disappointment in how well they longitudinally reflect disease progression. Some studies have reported small longitudinal effects (Glodzik-Sobanska et al., Neurobiol Aging, 2009; 30(5): 672-681; Kanai et al., Ann Neurol, 1998; 44(1): 17-26) while others did not find such evidence in either NL (Sunderland et al., Biol Psychiatry, 2004; 56(9): 670-676) or in AD (Tapiola et al., Neurosci Lett, 2000; 280(2): 119-122). In an earlier report, a panel of AD biomarkers were examined and it was shown that the longitudinal change in T-tau load (ventricle corrected), unlike the T-tau level or the Aβ1-42 or Aβ1-40 levels, was the only CSF biomarker to demonstrate longitudinal progression effects in MCI. The basis for using a correction for the CSF Tau level as opposed to the Aβ level, comes from the observations that tau is a brain derived protein and as such has higher ventricle to lumbar CSF levels. CSF measures that are not exclusively brain derived typically have higher lumbar than ventricle concentrations (Reiber, Clin Chim Acta, 2001; 310(2): 173-186). Moreover, the basis for the correction considers that as the volume of brain tissue is lost and tau released into the extracellular fluid, there is a proportional increase in the CSF volume of the brain, thus keeping the total intracranial volumes of brain and water constant. Thus, dilution correction accounts for the increase in CSF volume that accompanies the decrease in brain volume, the presumed basis for the amount of tau released. These dilution effects potentially null the apparent longitudinal increases in T-tau concentrations. Previous results showed that adjusting for the ventricular enlargement of AD in effect corrects for the dilution of tau, and improves the detection of the longitudinal change in CSF tau (de Leon et al., Neurosci Lett, 2002; 333(3): 183-186).

### SUMMARY OF THE INVENTION

Detection of cerebrospinal fluid (CSF) tau changes over time has been proposed as a biomarker of Alzheimer's Disease. The present invention demonstrates that after CSF dilution correction, the CSF T-tau can detect the longitudinal effects in preclinical Alzheimer's Disease. The present invention further demonstrates that longitudinal T-tau load is a useful biomarker for the presymptomatic detection of brain damage as related to Alzheimer's Disease.

In a first aspect, the present invention provides a method for diagnosing a neurological disease in a subject featuring
(a) obtaining a cerebrospinal fluid (CSF) sample from a subject;
(b) providing a cerebrospinal fluid (CSF) correction factor for CSF obtained from a subject for at least one biomarker; and
(c) determining whether the biomarker is present in elevated amounts or concentrations in the cerebrospinal fluid (CSF) sample.

The neurological disease may be, for instance, impaired cognition or dementia such as Alzheimer's Disease (AD) or Mild Cognitive Impairment (MCI). The biomarker may be a tau protein such as P-tau₂₃₁ or a total of all or multiple tau proteins or an amyloid beta (Aβ) such as Aβ₄₂ or Aβ₄₀. The biomarker may be present in elevated amounts or concentrations in the cerebrospinal fluid (CSF) sample that 5%, 10%, 20%, 25%, 30%, 40%, 50%, 60%, 70%, 75%, 80%, 90% or more higher, or two or three or four or five or six or more times higher than the amounts or concentrations in the a cerebrospinal fluid (C SF) sample obtained from a normal control subject or in a cerebrospinal fluid (C SF) sample obtained from the very same subject at an earlier point in time or the amounts or concentrations represented by a reference value. In some embodiments, the biomarker such as a tau protein or an amyloid beta is measured by a quantitative method such as an immunological or biochemical assay specific for the biomarker. In yet other particular embodiments, the biomarker is measured by an enzyme-linked immunosorbent assay (ELISA), a Western blot assay, a Northern blot assay, and a Southern blot assay. The amount or concentration of the biomarker may be measured repeatedly, for instance, twice, ten times, twenty times, fifty times, seventy-five times, one hundred times, two hundred times or more. Likewise, the amount or concentration of the biomarker may be measured at regular or semi-regular intervals, for instance, every 5 or 10 days, every week, biweekly, semi-weekly, monthly, every two months, quarterly, twice a year, yearly, every two years, every three years, etc. As such, the invention provides for mapping, charting or comparing the amounts or concentration of the biomarker over longitudinal periods of time.

In a second aspect, the present invention provides a method for screening for or assessing the risk of developing a neurological disease in a subject featuring
(a) obtaining a cerebrospinal fluid (CSF) sample from a subject;
(b) providing a cerebrospinal fluid (CSF) correction factor for CSF obtained from a subject for at least one biomarker; and
(c) determining whether the biomarker is present in elevated amounts or concentrations in the cerebrospinal fluid (CSF) sample.

The neurological disease may be, for instance, impaired cognition or dementia such as Alzheimer's Disease (AD) or Mild Cognitive Impairment (MCI). The biomarker may be a P-tau such as P-tau₂₃₁ or multiple or a total of all or multiple tau proteins or an Aβ such as Aβ₄₂ or Aβ₄₀ The biomarker may be present in elevated amounts or concentrations in the cerebrospinal fluid (CSF) sample that 5%, 10%, 20%, 25%, 30%, 40%, 50%, 60%, 70%, 75%, 80%, 90% or more higher, or two or three or four or five or six or more times higher than the amounts or concentrations in the a cerebrospinal fluid (CSF) sample obtained from a normal control subject or in a cerebrospinal fluid (CSF) sample obtained from the very same subject at an earlier point in time or the amounts or concentrations represented by a reference value. In some embodiments, the biomarker such as a tau protein or an amyloid beta is measured by a quantitative method such as an immunological or biochemical assay specific for the biomarker. In yet other particular embodiments, the biomarker is measured by an enzyme-linked immunosorbent assay (ELISA), a Western blot assay, a Northern blot assay, and a Southern blot assay. The amount or concentration of the biomarker may be measured repeatedly, for instance, twice, ten times, twenty times, fifty times, seventy-five times, one hundred times, two hundred times or more. Likewise, the amount or concentration of the biomarker may be measured at regular or semi-regular intervals, for instance, every 5 or 10 days, every week, biweekly, semi-weekly, monthly, every two months, quarterly, twice a year, yearly, every two years, every three years, etc. As such, the invention provides for mapping, charting or comparing the amounts or concentration of the biomarker over longitudinal periods of time.

In a third aspect, the present invention provides a method for monitoring progression of a neurological disease in a subject featuring
(a) obtaining a cerebrospinal fluid (CSF) sample from a subject;
(b) providing a cerebrospinal fluid (CSF) correction factor for CSF obtained from a subject for at least one biomarker; and
(c) determining whether the biomarker is present in elevated amounts or concentrations in the cerebrospinal fluid (CSF) sample.

The neurological disease may be, for instance, impaired cognition or dementia such as Alzheimer's Disease (AD) or Mild Cognitive Impairment (MCI). The biomarker may be a P-tau such as P-tau₂₃₁ or a total of all or multiple tau proteins or an Aβ such as Aβ₄₂ or Aβ₄₀. The biomarker may be present in elevated amounts or concentrations in the cerebrospinal fluid (CSF) sample that 5%, 10%, 20%, 25%, 30%, 40%, 50%, 60%, 70%, 75%, 80%, 90% or more higher, or two or three or four or five or six or more times higher than the amounts or concentrations in the a cerebrospinal fluid (CSF) sample obtained from a normal control subject or in a cerebrospinal fluid (CSF) sample obtained from the very same subject at an earlier point in time or the amounts or concentrations represented by a reference value. In some embodiments, the biomarker such as a tau protein or an amyloid beta is measured by a quantitative method such as an immunological or biochemical assay specific for the biomarker. In yet other particular embodiments, the biomarker is measured by an enzyme-linked immunosorbent assay (ELISA), a Western blot assay, a Northern blot assay, and a Southern blot assay. The amount or concentration of the biomarker may be measured repeatedly, for instance, twice, ten times, twenty times, fifty times, seventy-five times, one hundred times, two hundred times or more. Likewise, the amount or concentration of the biomarker may be measured at regular or semi-regular intervals, for instance, every 5 or 10 days, every week, biweekly, semi-weekly, monthly, every two months, quarterly, twice a year, yearly, every two years, every three years, etc. As such, the invention provides for mapping, charting or comparing the amounts or concentration of the biomarker over longitudinal periods of time.

In a fourth aspect, the present invention provides a method for assessing efficacy of a therapy for a neurological disease in a subject featuring
(a) obtaining a cerebrospinal fluid (CSF) sample from a subject;
(b) providing a cerebrospinal fluid (CSF) correction factor for CSF obtained from a subject for at least one biomarker; and
(c) determining the amount or concentration of the biomarker in the cerebrospinal fluid (CSF) sample.

The neurological disease may be, for instance, impaired cognition or dementia such as Alzheimer's Disease (AD) or Mild Cognitive Impairment (MCI). The biomarker may be a P-tau such as P-tau₂₃₁ or a total of all or multiple tau proteins or an Aβ such as Aβ₄₂ or Aβ₄₀. The biomarker may be present in elevated amounts or concentrations in the cerebrospinal fluid (CSF) sample that 5%, 10%, 20%, 25%, 30%, 40%, 50%, 60%, 70%, 75%, 80%, 90% or more higher, or two or three or four or five or six or more times higher than the amounts or concentrations in the a cerebrospinal fluid (CSF) sample obtained from a normal control subject or in a cerebrospinal fluid (CSF) sample obtained from the very same subject at an earlier point in time or the amounts or concentrations represented by a reference value. The method may further feature comparing the amount or concentration of the biomarker in the cerebrospinal fluid (C SF) sample with the amount or concentration of the biomarker in a cerebrospinal fluid (CSF) sample obtained from the subject at an earlier point in time. In some embodiments, the biomarker such as a tau protein or an amyloid beta is measured by a quantitative method such as an immunological or biochemical assay specific for the biomarker. In yet other particular embodiments, the biomarker is measured by an enzyme-linked immunosorbent assay (ELISA), a Western blot assay, a Northern blot assay, and a Southern blot assay. The amount or concentration of the biomarker may be measured repeatedly, for instance, twice, ten times, twenty times, fifty times, seventy-five times, one hundred times, two hundred times or more. Likewise, the amount or concentration of the biomarker may be measured at regular or semi-regular intervals, for instance, every 5 or 10 days, every week, biweekly, semi-weekly, monthly, every two months, quarterly, twice a year, yearly, every two years, every three years, etc. As such, the invention provides for mapping, charting or comparing the amounts or concentration of the biomarker over longitudinal periods of time.

In an fifth aspect, the present invention provides a method of identifying a subject suffering from a neurological disease, for instance, mild cognitive impairment or a dementia such as Alzheimer's Disease, that may be successfully treated by an agent that affects levels of a biomarker such as a tau protein or an amyloid beta featuring
(a) obtaining a cerebrospinal fluid (CSF) sample from a subject;
(b) providing a cerebrospinal fluid (CSF) correction factor for CSF obtained from a subject for at least one biomarker; and
(c) determining the amount or concentration of the biomarker in the cerebrospinal fluid (CSF) sample.

The neurological disease may be, for instance, impaired cognition or dementia such as Alzheimer's Disease (AD) or Mild Cognitive Impairment (MCI). The biomarker may be a P-tau such as P-tau₂₃₁ or a total of all or multiple tau proteins or an Aβ such as Aβ₄₂ or Aβ₄₀. The biomarker may be present in elevated amounts or concentrations in the cerebrospinal fluid (CSF) sample that 5%, 10%, 20%, 25%, 30%, 40%, 50%, 60%, 70%, 75%, 80%, 90% or more higher, or two or three or four or five or six or more times higher than the amounts or concentrations in the a cerebrospinal fluid (CSF) sample obtained from a normal control subject or in a cerebrospinal fluid (CSF) sample obtained from the very same subject at an earlier point in time or the amounts or concentrations represented by a reference value. The method may further feature comparing the amount or concentration of the biomarker in the cerebrospinal fluid (C SF) sample with the amount or concentration of the biomarker in a cerebrospinal fluid (CSF) sample obtained from the subject at an earlier point in time. In some embodiments, the biomarker such as a tau protein or an amyloid beta is measured by a quantitative method such as an immunological or biochemical assay specific for the biomarker. In yet other particular embodiments, the biomarker is measured by an enzyme-linked immunosorbent assay (ELISA), a Western blot assay, a Northern blot assay, and a Southern blot assay. The amount or concentration of the biomarker may be measured repeatedly, for instance, twice, ten times, twenty times, fifty times, seventy-five times, one hundred times, two hundred times or more. Likewise, the amount or concentration of the biomarker may be measured at regular or semi-regular intervals, for instance, every 5 or 10 days, every week, biweekly, semi-weekly, monthly, every two months, quarterly, twice a year, yearly, every two years, every three years, etc. As such, the invention provides for mapping, charting or comparing the amounts or concentration of the biomarker over longitudinal periods of time.

The present invention relates to methods of screening, diagnosing or prognosing a neurological disorder such as mild cognitive impairment or Alzheimer's Disease in a subject. The invention further relates to identifying a subject at risk for developing a neurological disorder such as mild cognitive impairment or Alzheimer's Disease or for monitoring the effect of therapy administered to a subject suffering from a neurological disorder such as mild cognitive impairment or Alzheimer's Disease. The invention further relates to methods of screening a subject for elevated levels of a biomarker in cerebrospinal fluid as a means of determining whether that subject has, or is prone to developing, a neurological disorder such as mild cognitive impairment or Alzheimer's Disease. This biomarker may also be used as a means of assessing the effectiveness of therapy in subjects being treated for a neurological disorder such as mild cognitive impairment or Alzheimer's Disease. Thus, the present procedures for screening or diagnosing subjects with such conditions are minimally invasive allow for rapid and sensitive screening. The present methods are also useful for identifying subjects suffering from a neurological disorder such as mild cognitive impairment or Alzheimer's Disease who may be responsive to treatment with therapeutic agents that may lower brain levels of or concentrations of certain biomarkers such as, for instance, a tau protein or an amyloid beta. In addition, because Aβ₁₋₄₀ is known to be neurotoxic (like Aβ₁₋₄₂), lowering the levels of both Aβ₁₋₄₂ as well as, Aβ₁₋₄₀ in the brain may be beneficial in treating a neurological disorder featuring impaired cognition or dementia such as mild cognitive impairment or Alzheimer's Disease. The invention further relates to pharmaceutical compositions containing an agent that reduces brain tau or amyloid beta levels in the brain. The invention also relates to screening methods that aid in the identification of novel agents that function through any one of the above-noted mechanisms for use in the treatment of a neurological disorder such as mild cognitive impairment or Alzheimer's Disease.

U.S. Pat. No. 8,128,907, the disclosure of which is hereby incorporated by reference in its entirety, describes methods for providing a cerebrospinal fluid (CSF) correction factor for CSF obtained from a patient suffering from a neurological disease for at least one biomarker such as, for instance, a Tau protein or an amyloid beta (Aβ). These methods are useful for each aspect of the present invention and feature the steps of:
(a) measuring levels of said at least one biomarker in samples of CSF obtained from & the patient;
(b)-providing semi-automated measurements of the ventricular system using quantitative anatomical protocols;
(c) correcting the concentration dilution level in the CSF of said at least one biomarker measured in step a) by using a computer algorithm that analyzes volume and longitudinal changes in CSF measurements obtained in step (b); and
(d) providing the CSF correction factor for level of concentration of said at least one biomarker due to increased ventricular volume of CSF caused by an increase in ventricular size.
   The biomarker may be, for instance, a tau protein such as P-tau₂₃₁, Aβ₄₂, or Aβ₄₀ or any combination of the same.

Similarly, these methods for providing a cerebrospinal fluid (CSF) correction factor for CSF obtained from a patient suffering from a neurological disease for at least one biomarker such as, for instance, a Tau protein or an amyloid beta (Aβ), useful for each aspect of the present invention may feature the steps of:
(a) measuring levels of biomarkers in CSF samples obtained from the patient;
(b) providing semi-automated measurements of the ventricular system using quantitative anatomical protocols; and
(c) correcting the concentration dilution level in the CSF of said at least one biomarker tested for in step (a) by a using a computer algorithm that analyzes the volume and longitudinal changes in CSF measurements obtained in step (b);and
(d) providing the CSF correction factor for the concentration of said at least one biomarker due to increased ventricular volume of CSF.

The biomarker may be, for instance, a tau protein such as P-tau₂₃₁, Aβ₄₂, or Aβ₄₀ or any combination of the same.

Likewise, these methods for providing a cerebrospinal fluid (CSF) correction factor for CSF obtained from a patient suffering from a neurological disease for at least one biomarker such as, for instance, a Tau protein or an amyloid beta (Aβ), useful for each aspect of the present invention may feature the steps of:
(a) measuring the ventricular system of the brain by scans;
(b) measuring levels of a biomarker selected from the group consisting of a Tau protein and an amyloid beta (Aβ) peptide in CSF that has been extracted within a predetermined amount of time before or after the scanning in step (a);
(c) correcting a concentration dilution level in the CSF of said at least one biomarker tested for in step (b) by using a computer algorithm that compares the measurements from the scans in step (a) with average values for a particular head size to determine ventricular size; and
(d) providing the cerebrospinal fluid (CSF) correction factor for CSF obtained from a patient suffering from a neurological disease.

The biomarker may be, for instance, a tau protein such as P-tau₂₃₁, Aβ₄₂, or Aβ₄₀ or any combination of the same.

Also, these methods for providing a cerebrospinal fluid (CSF) correction factor for CSF obtained from a patient suffering from a neurological disease for at least one biomarker such as, for instance, a Tau protein or an amyloid beta (Aβ), useful for each aspect of the present invention may feature the steps of:
(a) collecting longitudinal data of scans;
(b) co-registering a baseline scan and a follow-up scan of images;
(c) constructing a border region B and two interior regions I_{bas}, I_{fol} for the entire brain and for each predetermined anatomical subvolume of interest;
(d) computing signal loss and volume loss of the brain to derive a volume of CSF in the brain, and
(e) providing an output of a correction factor for correcting a tested level of at least one biomarker in the CSF.

The biomarker may be, for instance, a tau protein such as P-tau₂₃₁, Aβ₄₂, or Aβ₄₀ or any combination of the same.

### BRIEF DESCRIPTION OF THE DRAWINGS

FIG. 1 is a flowchart providing an overview of a method for correcting for concentration of a biomarker in the cerebrospinal fluid.

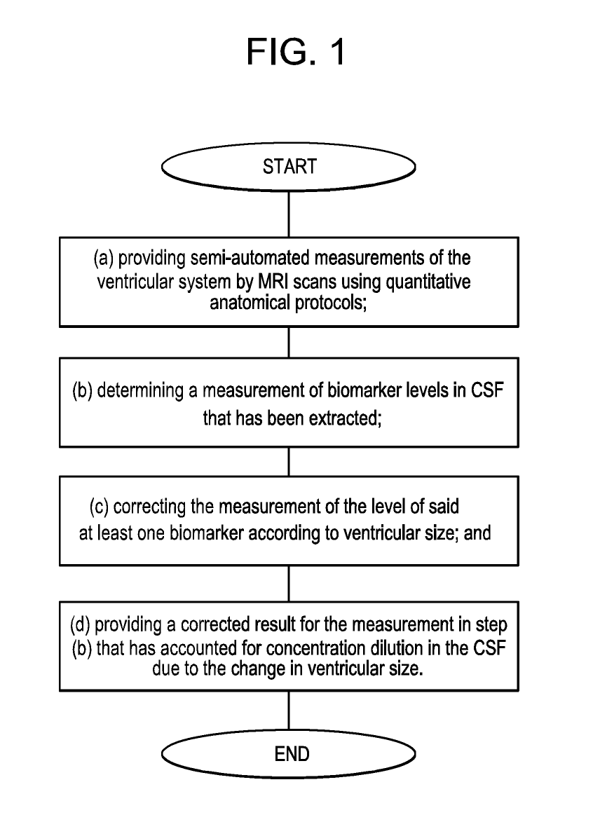

FIG. 2 is a flowchart providing an overview of a method for correcting for concentration of a biomarker in the cerebrospinal fluid.

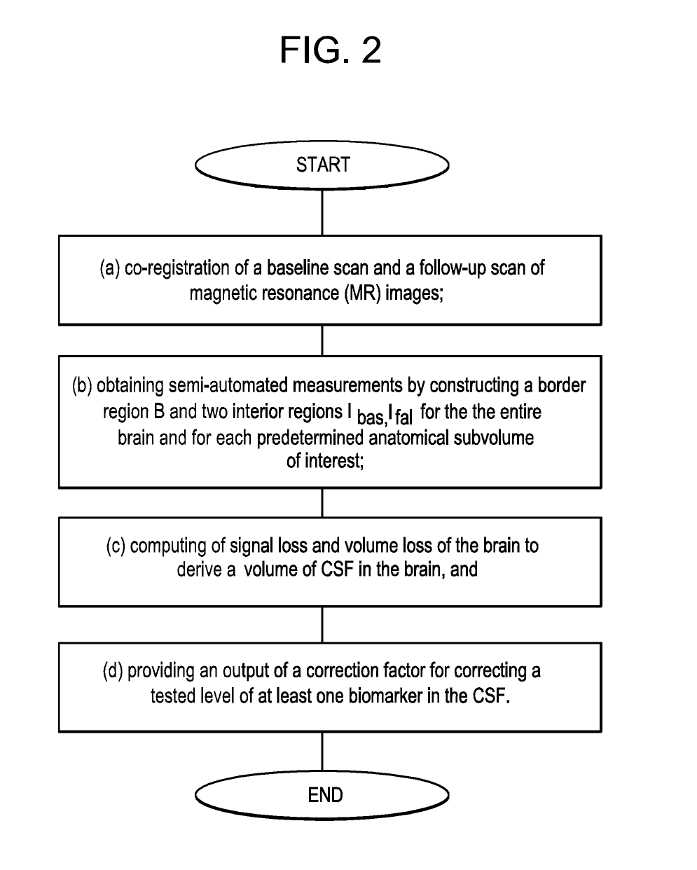

FIGS. 3A, 3B, and 3C plot the results that demonstrate the efficacy of the correction method based on ventricular volume of cerebrospinal fluid (V-CSF). Data are from two groups of elderly subjects whose cognitive status and CSF assays were collected for up to 10 years since their baseline exam. At baseline each subject was cognitively normal (NL). The first group (left bars in each chart) shows data for those who remained cognitively stable at the final exam. The second group (right bars in each chart) shows data for those were diagnosed with either MCI or AD at the final exam. The two whiskers show the range of values (the minimum and the maximum) and the dot indicated the median value. FIG. 3A shows the distribution of annual change rate (in percents) of T-tau levels. While the median change in T-tau levels is larger in the Decline group, there is a considerable overlap and statistical tests fail to demonstrate significance. FIG. 3B plots the distribution of annual changes (in percents) in ventricular volume for both groups. The ventricles become enlarged at a faster rate in the Decline group, but there is a substantial overlap across the two groups. FIG. 3C plots the distribution of annual changes (in percents) in T-tau load defined as the product of T-tau level and the corresponding ventricle volume. The overlap between the two groups is clearly lower than in FIGS. 3A and 3B. The star symbol indicates that statistical tests demonstrated significant group difference in annual changes for T-tau load, in spite of the lack of statistical significance in T-tau level.

FIG. 4A and FIG. 4B demonstrate correcting cerebrospinal fluid (CSF) biomarker data using the total volume of intracranial CSF (IC-CSF) instead of using the ventricle volume for correction. IC-CSF is the combined CSF in the ventricles and in the extra-ventricular spaces that surround the brain. FIG. 4A is identical to FIG. 3A, and it shows the distribution of annual change rate of T-tau levels in the two groups of elderly subjects. FIG. 4B plots the distribution of annual changes in the total volume of intracranial CSF volume for both groups. While IC-CSF grows at a faster rate in the Decline group, there is a large overlap across the two groups. FIG. 4C plots the distribution of annual changes in T-tau load defined as the product of T-tau levels and corresponding IC-CSF. The overlap between the two groups is clearly lower than in FIGS. 4A and 4B. As in the method shown in FIG. 3, statistical tests demonstrated a significant difference in annual changes for T-tau load computed using IC-CSF.

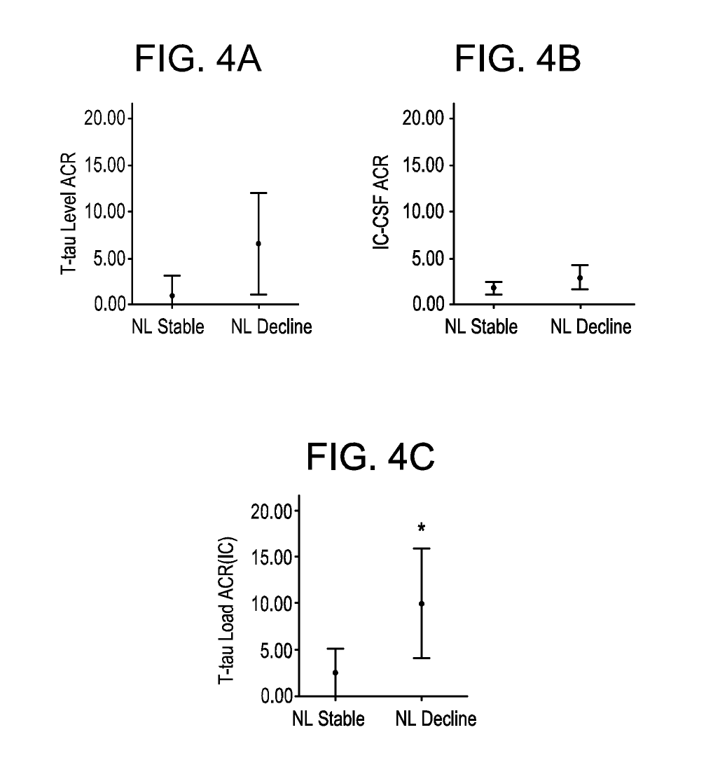

### DETAILED DESCRIPTION OF THE INVENTION

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entirety.

In accordance with the present invention there may be employed conventional molecular biology, microbiology, and recombinant DNA techniques within the skill of the art. Such techniques are explained fully in the literature. See, e.g., Sambrook et al., “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. PHames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

### Definitions

The terms used herein have the meanings recognized and known to those of skill in the art, however, for convenience and completeness, particular terms and their meanings are set forth below. Mild cognitive impairment, dementia and Alzheimer's Disease are diagnoses defined in the Diagnostic and Statistical Manual of Mental Disorders (DSM IV TR) classification system.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to prevent, and preferably reduce by at least about 25 percent, more preferably by at least 50 percent, most preferably by at least 90 percent, a clinically significant change in a feature of pathology such as for example, elevated blood pressure, fever, or white cell count, as may attend its presence and activity. As related to the present invention, the term may also mean an amount sufficient to ameliorate or reverse one or more symptoms associated with cognitive impairment, dementia or Alzheimer's Disease.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted.

“Subject” or “patient” refers to a mammal, preferably a human, in need of treatment for a condition, disorder or disease.

An “antibody” is any immunoglobulin, including antibodies and fragments thereof, that binds a specific epitope. Such an antibody that binds a specific epitope is said to be “immunospecific.” The term encompasses “polyclonal,” “monoclonal,” and “chimeric” antibodies, the last mentioned described in further detail in U.S. Pat. Nos. 4,816,397 and 4,816,567. Commonly used carriers that are chemically coupled to peptides include bovine or chicken serum albumin, thyroglobulin, and other carriers known to those skilled in the art. The coupled peptide is then used to immunize the animal (e.g, a mouse, rat or rabbit). The “chimeric antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397) The antibody may be a human or a humanized antibody. The antibody may be a single chain antibody. (See, e.g., Curiel et al., U.S. Pat. No. 5,910,486 and U.S. Pat. No. 6,028,059). The antibody may be prepared in, but not limited to, mice, rats, rabbits, goats, sheep, swine, dogs, cats, or horses. As used herein, the term “single-chain antibody” refers to a polypeptide comprising a V_{H} region and a V_{L} region in polypeptide linkage, generally linked via a spacer peptide (e.g., [Gly-Gly-Gly-Gly-Ser]ₓ), and which may comprise additional amino acid sequences at the amino- and/or carboxy-termini. For example, a single-chain antibody may comprise a tether segment for linking to the encoding polynucleotide. As an example, a scFv (single chain fragment variable) is a single-chain antibody. Single-chain antibodies are generally proteins consisting of one or more polypeptide segments of at least 10 contiguous amino acids substantially encoded by genes of the immunoglobulin superfamily (e.g., see The Immunoglobulin Gene Superfamily, A. F. Williams and A. N. Barclay, in Immunoglobulin Genes, T. Honjo, F. W. Alt, and T. H. Rabbitts, eds., (1989) Academic Press: San Diego, Calif., pp. 361-387, which is incorporated herein by reference), most frequently encoded by a rodent, non-human primate, avian, porcine, bovine, ovine, goat, or human heavy chain or light chain gene sequence. A functional single-chain antibody generally contains a sufficient portion of an immunoglobulin superfamily gene product so as to retain the property of binding to a specific target molecule, typically a receptor or antigen (epitope).

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule. Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Screening” or “Diagnosis” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

“Biomarker” refers to any molecule or agent such as a protein or peptide that may be present in elevated or reduced amounts or concentrations in a biological sample or tissue in subject suffering from a particular disease, disorder or condition. The term specifically includes the naturally occurring molecule or agent such as a protein or peptide as well as variants, analogs, homologs, derivatives and fragments thereof. In some instances the variants, analogs, or homologs may have about 75%, 80%, 90%, 95% or 99% or more sequence identity to the naturally occurring molecule. In some instances the variants, analogs, or homologs may display at least 10%, 25%, 50%, 100% or 200% of the biological activity of the naturally occurring molecule or agent.

“Derivative” refers to the chemical modification of molecules, either synthetic organic molecules or proteins, nucleic acids, or any class of small molecules such as fatty acids, or other small molecules that are prepared either synthetically or isolated from a natural source, such as a plant, that retain at least one function of the active parent molecule, but may be structurally different. Chemical modifications may include, for example, replacement of hydrogen by an alkyl, acyl, or amino group. It may also refer to chemically similar compounds which have been chemically altered to increase bioavailability, absorption, or to decrease toxicity. A derivative polypeptide is one modified by glycosylation, pegylation, or any similar process that retains at least one biological or immunological function of the polypeptide from which it was derived.

“Amyloid” describes various types of protein aggregations that share specific traits when examined microscopically. Amyloid is typically identified by a change in the fluorescence intensity of planar aromatic dyes such as Thioflavin T or Congo Red. This is generally attributed to the environmental change as these dyes intercolate between β-strands. The amyloid fold is characterized by a cross-β sheet quaternary structure, that is, a monomeric unit contributes a β strand to a β sheet which spans across more than one molecule. While amyloid is usually identified using fluorescent dyes, stain polarimetry, circular dichroism, or FTIR (all indirect measurements), the “gold-standard” test to see if a structure is amyloid is by placing a sample in an X-ray diffraction beam; there are two characteristic scattering bands produced at 4 and 10 angstroms each, corresponding to the interstrand distances in the β sheet structure. The amyloid protein disclosed in the present application refers to amyloid β, as described below.

“Amyloid beta,” “Aβ,” “β-amyloid” or “amyloid beta peptide” is a physiological product normally released from the amyloid beta protein precursor (βAPP or APP) through β and γ secretase cleavage and consists of two 40 and 42 amino acid peptides, usually abbreviated as Aβ₄₀ and Aβ₄₂, respectively (Selkoe, J. Clin. Invest. (1992); 110:1375-1381). The 42 amino acid amyloid beta peptide (Aβ₄₂) is more hydrophobic & “sticky” (and hence aggregates more readily) than the 40 amino acid amyloid beta peptide (Aβ₄₀), and as such may play a greater role in the pathogenesis of Alzheimer's disease, due to its increased tendency to form insoluble fibrils and increased neurotoxicity. Thus, under certain circumstances, as in Alzheimer's disease (AD), brain levels of these peptides increase dramatically, which can lead to the oligomerization of the peptides and eventually to the formation of insoluble fibrillar aggregates, which deposit in senile plaques. In the present application, “amyloid β₄₀” is used interchangeably with “Aβ₄₀”, Aβ₄₀ and Aβ₁₋₄₀, and “amyloid β₄₂” is used interchangeably with “Aβ₄₂,” Aβ₄₂ and Aβ₁₋₄₂. The nucleic acid and amino acid sequences for amyloid beta precursor protein and Aβ₄₀ and Aβ₄₂ are found in SEQ ID NOS: 1, 2, 3, 4 and 5. SEQ ID NO: 1 is the nucleic acid sequence encoding human amyloid beta precursor protein; SEQ ID NO: 2 is the nucleic acid encoding human Aβ₄₀ peptide; SEQ ID NO: 3 is the amino acid sequence of human Aβ₄₀ peptide; SEQ ID NO: 4 is the nucleic acid encoding human Aβ₄₂, SEQ ID NO: 5 is the amino acid sequence of human Aβ₄₂ peptide.

“Fragment” refers to either a protein or polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide.

“Surrogate biomarker” or “biomarker” as used herein, refers to a molecule, the existence and levels of which are causally connected to a complex biological process, and reliably captures the state of the process. Furthermore, a surrogate biomarker, to be of practical importance, must be present in samples that can be obtained from individuals without endangering their physical integrity or well-being, preferentially from biological fluids such as blood, urine, saliva or tears.

“Monitoring the effect of therapy administered to a subject” as used herein refers to a situation wherein a subject is being treated for a neurological disease using at least one drug, and it is desirable to determine whether there are any benefits to such treatment. The effects of such therapy may be determined by evaluating the subject using standard procedures for such evaluation by a trained physician, preferably a psychiatrist or clinical psychologist. Well known rating scales for cognitive function are used.

Twenty-six cognitively normal subjects (NL) who received 2 sets of CSF and MRI exams, once at baseline and once at 3 year follow-up and a minimum of three annual follow-up clinical and neuropsychological evaluations for up to 10 years. Seven subjects declined to mild cognitive impairment (MCI) in 4.5 years, and 3 of them declined to AD (average 6 years after baseline). Intracranial CSF T-tau load (ICV) and ventricular CSF T-tau load (V) was calculated by multiplying the T-tau concentration by the CSF volume (either by the V-CSF (ventricle CSF volume) or the IC-CSF (intracranial CSF volume)) and dividing by 1000. The annual change rate (ACR) of T-tau load (ICV) and T-tau load (V) were significantly higher in NL decline group, neither ACR of T-tau level or CSF volume were significant. Delayed memory score (PARD) was negatively correlated with the ACR for the T-tau load (ICV) and T-tau load (V).

### Cerebrospinal Fluid Biomarker Correction Factors

The present invention uses methods for correcting biomarker dilution in ventricular CSF that greatly reduces the time needed to make the corrections, thus permitting relatively inexpensive and accurate diagnosis and monitoring of biomarkers that were previously cost-prohibitive to be used as a routine test. The methods used may correct any biomarker found in CSF, and not just the markers associated with MCI and Alzheimer's Disease.

The methods permit fast and accurate detection of ventricular size so as to adjust the level of biomarkers according to the CSF volume and reduce the time to make a correction to a matter of minutes. The methods do not require highly trained specialists in brain anatomy to study the scans and provide a relatively fast and inexpensive way to screen for, diagnose, monitor or assess risk for developing MCI or Alzheimer's Disease, both for those who show signs of such illnesses, as well as for those who do not. The methods may be combined with MRI and any number of CSF markers to improve the sensitivity and specificity for the prediction of cognitive decline.

The methods allow detecting and intervening relatively early to slow or inhibit the progression of certain brain disorders before a patient's illness has advanced to a stage that produces a severe impact. These methods provide a significant advance in the ability to rapidly process ventricular volume of the brain. These methods allow the correction of the biomarker levels in CSF by using the MRI or CT scans, much more quickly than previously so as to evaluate disorders that may be detected by identifying and quantifying biomarkers in the CSF.

Progressive hippocampal and entorhinal cortex (EC) atrophy, early sites of Alzheimer pathology, can be best predicted by elevated CSF P-tau₂₃₁ levels that are corrected according to the improved diagnostic accuracy of the present invention. Progressive neocortical atrophy can best be predicted by reductions in CSF Aβ₄₀ and Aβ₄₂ levels that are corrected according to the improved diagnostic accuracy of the present invention. These methods may be used for any brain pathology or other biomarker found in CSF whose concentration may be affected by a change in ventricular size. These methods may also be used to track atrophy in other parts of the brain. Blood samples from a patient can be tested for the presence of biomarkers in conjunction with the corrected levels of biomarkers in the CSF to provide a more accurate correlate of blood values and improve diagnostic understanding of a patient's illness or its progression.

These methods for correcting measurements of at least one biomarker in cerebrospinal fluid (CSF) feature (a) providing semi-automated measurements of the ventricular system by MRI scans using quantitative anatomical protocols; (b) determining a measurement of biomarker levels in CSF that has been extracted; (c) correcting the measurement of the level of said at least one biomarker according to the ventricular size; and (d) providing a corrected result of the measurement determined in step (b), said corrected result accounting for concentration dilution due to the increase in ventricular size (which can be caused by the decrease in the size of the damaged brain). It is known from using MRI that the hippocampus and EC are affected earliest in the course of Alzheimer's disease. Thus, said correlated CSF corrected results in combination with MRI improve the diagnostic sensitivity and specificity.

The change that can be measured is the change in ventricular size at one time point or over time, and is used to adjust the amount of biomarker measured in the CSF. The adjusted amount of a particular biomarker level in the CSF is due to the correlated MRI scan of the brain and measurement of the ventricular volume. The MRI corrected CSF biomarker can then be used to assess cross sectional deviations from reference norms and longitudinal change.

In addition to measuring the ventricular size from MRI scans, it is possible using the semi-automated procedures described herein to measure the change of other regions of the brain, including the entorhinal cortex, hippocampus, and other potentially vulnerable regions. The hippocampus and entorhinal cortex are predictors of progressive memory loss and cognitive decline in Alzheimer's Disease (de Leon, et al., American Journal of Neuroradiology 1993; 14: 897-906; de Leon, et al., PNAS USA, 2002; 98: 10966-10971).

These useful measurements for early diagnosis and prediction of future Alzheimer's disease may feature using the CSF corrected biomarker tau. In some instances featuring correcting certain specific biomarkers for Alzheimer's Disease and MCI, the above-mentioned steps (c) and (d) may be modified to feature (c) correcting the measured level of tau and related proteins (P-tau₂₃₁) and fragment of amyloid beta found in the CSF by an algorithm that analyzes the measurements from the MRI scans of ventricular size; and (d) providing a corrected result that has accounted for concentration dilution in the CSF due to the increase in ventricular size (caused by the decrease in the size of the brain).

These methods are particularly valuable for correcting biomarker levels for the aforementioned illnesses because neuropathology studies show that damage to the hippocampus and entorhinal cortex, the brain's major memory processing center, occurs early in the course of Alzheimer's Disease. Thus more biomarker proteins are sequestered in the CSF compartment of the brain. As a result, an accurate measurement of increased ventricular size rapidly and inexpensively provides a significant improvement over the prior art.

The determining of CSF biomarker levels in step (b) may include a time limitation (plus or minus a predetermined amount of time) before or after the MRI a level of at least one predetermined biomarker.

In some instances, Aβ₄₂ levels in CSF may be corrected by (a) MRI scans using quantitative anatomical protocols to provide measurements of the ventricular system, and that may optionally include measuring the EC, hippocampus, and vulnerable neocortical regions of the brain; (b) testing CSF that has been extracted plus or minus a predetermined time period before or after the MRI for CSF Aβ₄₂ levels; (c) correcting the measurement of the level of Aβ₄₂ in the CSF by an algorithm that analyzes the measurements from the MRI scans of at least the ventricular size; and (d) providing a corrected result that has accounted for concentration dilution due to the increase in ventricular size.

In some instances both P-tau₂₃₁ and Aβ₄₂ levels may be corrected by: (a) MRI scans using quantitative anatomical protocols to provide measurements of the ventricular system; (b) testing CSF that has been extracted within a predetermined amount of time before or after the MRI for P-tau₂₃₁ and CSF Aβ₄₂ levels; (c) correcting the measurement of the level of Aβ₄₂ in the CSF by an algorithm that analyzes the measurements from the MRI scans of at least the ventricular system; and (d) providing a corrected result that has accounted for concentration dilution due to the increase in ventricular size (caused by the decrease in the brain tissue volume).

In the early stages of Alzheimer's the brain, particularly the hippocampus, decreases in size, and the volume of the ventricle increases proportionally. The amount of CSF fluid increases as the brain attempts to maintain its structural integrity within the bony calvarium. Thus, with the decrease in hippocampal and EC sizes being one early indicator consistent with Alzheimer's Disease, the brain increases the ventricular CSF volume. Accordingly, with each progressive stage of Alzheimer's (or other forms of dementia previously mentioned) the ventricular CSF volume increases and progressively dilutes the CSF derived biomarkers (reflecting the degenerative portions of the brain). This is one of the reasons why the levels, in particular of P-tau₂₃₁ and Aβ₄₂ in the CSF, do not appear to change as Alzheimer's Disease progresses. The amount of released P-tau₂₃₁ and Aβ₄₂ do increase, but the concentration of these biomarkers in CSF does not change because of the increased amounts of CSF in Alzheimer's patients. As the ventricular CSF increases, the concentration of biomarkers may remain the same even though there is an increase in the quantity of neurons and their tau protein that is affected and have been broken down and are introduced to the CSF for clearance.

The present invention is not limited to any particular type of MRI data but may include a single sequence, such as high resolution fast gradient recalled echo (GRE or MPRAGE) data. Such a high resolution sequence for anatomical work can be acquired at the baseline and follow up in any plane (sagittal, axial or coronal). An example of such a sequence may be defined as TR=35 ms, TE=9 ms, 60 degree flip angle, 256×192 acquisition matrix, 1.1 mm section thickness, 124 sections, 24 cm FOV, and a 1 NEX, for a total acquisition time of 12 minutes. In addition, the MRI data may be a multi-sequence including, for instance, two IR sequences, multiple echo MR sequences, etc., that may be used to segment the brain into cerebral gray matter, white matter and cerebrospinal fluid as known in the art, exemplified by Rusinek, et al., Radiology, 1991; 178: 109-114 and Rusinek et al., Investigative Radiology, 1993; 18928: 890-895, the contents of which are herein incorporated by reference in their entireties.

Measuring ventricular volume may be performed by CT scanning, followed by a summation of the individual section ventricular volumes so that the volume for each individual section is derived by a number of pixels within a user-defined cerebrospinal fluid range of attenuation coefficients.

Furthermore, there is a user-friendly method for rapid brain and CSF volume calculation using transaxial MRI images (Harris et al., Psychiatry Research, 1991; 40(1):61-8, the contents of which are herein incorporated by reference). Whole brain and CSF volumes are determined from a set of contiguous transaxial MRI images. A semi-automatic algorithm used a threshold guided edge follower, and subtraction of proton-weighted from T2-weighted images was used to highlight CSF.

In addition, MRI imaging data may be acquired using a spin-echo sequence in a single thick slice encompassing the head (Malko et al. American Journal of Neuroradiology, 1991; 12(2):371-4, the contents of which is incorporated herein by reference). The diagnostic MRI sequence may be provided for by obtaining axial FSE T2-weighted images, TR=7000 and TE=100 ms, FOV=20 cm, 16 echo trains, 256×256 matrix, 1 NEX, with 48 contiguous 3 mm slices, which take approximately 3.5 minutes to complete with the present technology.

While the algorithms could be incorporated into many different software packages, the algorithms have been implemented herein in a MIDAS software package developed in an NYU laboratory (Tsui et al., SHE Medical Imaging: Imaging Processing, Analyzing multi-modality Tomographic images and associated regions of interest with MIDAS, 2001; 4322: 1725034, the contents of which are herein incorporated by reference). MIDAS is a three-dimensional image analysis package with architecture enabling the implementation of highly interactive segmentation algorithms as add-on modules, which is particularly suited for segmentation, visualization and measure of the brain. The software application uses semi-automated procedures for (1) the volume of the CSF compartment that is known to increase with patient severity in Alzheimer's Disease, and (2) a novel method for measuring longitudinal changes in either CSF or in brain tissue. The increase in volume of the CSF and ventricular CSF levels for tau and amyloid beta are two-fold or greater higher than lumbar CSF levels. Therefore it is believed to dilute the protein level and provide inaccurate results.

Biomarker Dilution Factor: Semi-Automated CSF Volumes Estimated from T1-Weighted MRI Sequence

The software procedure for the CSF volume computation decomposes the MR image into three regions: the brain-CSF border B, the interior region containing CSF I, and the outer brain region O. A set of edge voxels is first constructed by thresholding the image at the gray level lower than 0.55 W, where W is the average white matter signal (The constant 0.55 was determined empirically using phantom studies). The set of voxels resulting from this step are denoted as M. The interior region I is obtained as a result of a “peel” operator. I=peel(M) and the outer region O is constructed as the complement of the operator grow (M). The border region B is then constructed as:

B=O−I.

All voxels within the region I are classified as the “pure CSF”. All voxels in the O region are classified as “brain tissue” voxels. Voxels within the border region B belong to the class of partially-volumed tissue. These voxels may contribute to segmentation error in a simpler image processing algorithm. The Midas software automatically decomposes each element B into fractional volumes of CSF and the brain based on their gray level and algebraic formulas. If the computation of CSF volume needs to be restricted to one or more regions of interest (ROI), the sets of I, O, and B are intersected with user-specific ROIs to yield regional estimates of the CSF and brain volumes.

Semi-Automated Procedures for Collecting Longitudinal Data from MRI Scans

The main image processing steps are (1) co-registration of baseline and follow-up MR images; (2) construction of border region B and two interior regions I_{bas} and I_{fol}, for the entire brain and for each anatomical subvolume of interest; and (3) computation of signal loss and volume loss of the brain.

The co-registration substeps (from step (1) above) may include (a) initial alignment of baseline scan to achieve standard orientation. In one embodiment, the pathologic angle is the standard angle, but the invention is not limited to this embodiment; (b) initial, rough co-registration by identifying three landmarks (superior colliculi, right and left optical tracts at their entry into the cranium) on baseline and follow up images. The software determines the transformation that maps the follow up to baseline locations. No resampling is performed at this stage; (c) exclusion of nonbrain tissues. While the intracranial structures are relatively rigid, the skin, scalp and muscles exhibit a substantial amount of plasticity. The automated software employs a region growing technique to segment out the brain parenchyma from the MR image; (d) iteratively refining the initial transformation matrix in step (b) to minimize the cost function defined as the variance of the ratio image. The accuracy is well under the voxel size. Voxels outside the brain paremchyma are excluded from this matching process; and (e) resampling the follow-up scan to match precisely the coordinate system of the baseline scan using the synch interpolation method. Both scans undergo one resampling transformation. Resampled and co-registered scans are then saved to disk.

The construction of the border region and interior region substeps (from step (2) above) may include (a) constructing a set of brain edge voxels by thresholding the image at the level of 0.55W, where W is the average white matter signal. (The constant 0.55 was determined empirically using phantom studies). The brain voxels resulting from this step may be denoted M_{bas} and M_{fol}; (b) obtaining interior regions as a result of a peel operator, (c) constructing region O₁ (outer border) as: grow (M_{bas} union M_{fol}); (d) constructing region O₂ (inner border) as peel (M_{bas} intersection M_{fol}); (e) constructing the border region B as O₁-O₂.

The stability and sensitivity of the methods is due to the decomposition of each brain subvolume into the border region B and the interior regions I_{bas} and I_{fol}. The MR signal intensity averaged over these interior regions is used to normalize the MR signal intensity across the two scans (to eliminate changes in system gain and attenuation). The border region B is a band around the brain edge over and above the shift between its location on baseline and followup scans. The border region is constructed automatically first for the entire brain, then it is intersected with brain subvolumes to yield regional borders.

The computation of the signal loss (described in step (3) above) and volume loss of the brain may be computed from the difference in MR signal in the border region of the baseline and the follow-up scans, after normalization by signal intensity in corresponding interior regions.

There is an increase in CSF volume in response to a loss (reduction) in brain volume. The CSF signal change may be converted to the CSF volume, and as the CSF volume increases, it may be expressed as either the absolute volume, or the percentage of the baseline CSF volume or the percentage of the baseline intracranial volume.

The dilution hypothesis was applied for the first time to a very important clinical distinction, characterizing biomarker progression among cognitively normal subjects. The dilution was estimated in two ways, by calculating the T-tau load using the ventricle and using the total intracranial CSF volume. The data show that the two measurements are equivalent and both demonstrate the longitudinal progression of the dilution corrected T-tau in normal subjects who show cognitive decline. Further confirming the progression effects significant longitudinal correlations between changes on declarative memory performance and the T-tau load change were observed. This is the first observation of longitudinal CSF T-tau effects in preclinical AD.

At cross-section, none of the T-tau measurements separated the NL-Stable from the NL-Decline groups. The differences in neuronal damage between normal aging and cognitively normal elderly destined to develop clinical features of AD may be subtle. This is supported by the absence of global atrophy effects using MRI and by a postmortem study that does not distinguish normal aging from mild clinical disease due to the variability and overlap in the tau pathology (Morris, et al., Neurology, 1996; 46(3): 707-719). This longitudinal data demonstrates robust progression effects providing clinically useful data.

In some embodiments, the present invention provides for tau or amyloid beta as a biomarker for a neurological disease. In accordance with the present invention, the tau or amyloid beta protein or fragments thereof, can be obtained using minimally invasive procedures. In a particular aspect, the invention provides for methods of screening, diagnosis or prognosis of a neurological disease in a subject, or for identifying a subject at risk for developing a neurological disease, or for monitoring the effect of therapy administered to a subject having a neurological disease. In a particular embodiment, the method comprises the steps of:
a. collecting a biological test sample from said subject;
b. analyzing said test sample for the presence of tau or amyloid beta levels; and
c. comparing the level of or concentration of tau or amyloid beta in the test sample with the level of or concentration of tau or amyloid beta in one or more persons free from a neurological disease, or with a previously determined reference range for tau or amyloid beta established from subjects free of a neurological disease.

While the tau or amyloid beta or fragment thereof may be measured in any bodily tissue sample from the subject, it is desirable to perform the measurement from a sample of cerebrospinal fluid (CSF). In this manner, the sample may be obtained by minimally invasive procedures. Thus, the determination of elevated levels of tau or amyloid beta in the sample from a patient who potentially has a neurological disease or who is prone to development of a neurological disease can be made and such measurement can be used as a surrogate marker for determination of a neurological disease.

The methods of the present invention provide for use of an antibody that binds to (is specific for) a tau protein or an amyloid beta, Aβ₄₀, Aβ₄₂ or fragments thereof and may be a monoclonal or polyclonal antibody specific for a tau or an amyloid beta, in particular, tau₂₃₁ pr amyloid β₁₋₄₀ or amyloid β₁₋₄₂. In a particular embodiment, the step of quantitatively measuring comprises testing a plurality of aliquots with a plurality of antibodies for the quantitative detection of a tau or an amyloid beta.

### Screening Methods for Measuring Tau and Amyloid Beta Levels

Antibodies to Tau or Amyloid beta for Therapeutic or Diagnostic Use

According to the present invention, tau and amyloid beta, including Aβ₄₀ and Aβ₄₂, as produced by a recombinant source, or through chemical synthesis, or isolated from natural sources; and derivatives, analogs and fragments thereof, including fusion proteins, may be used as an immunogen to generate antibodies that recognize the tau or amyloid beta, as exemplified below. Such antibodies include but are not limited to polyclonal, monoclonal, chimeric including humanized chimeric, single chain, Fab fragments, and a Fab expression library. The anti-tau and anti-amyloid beta antibodies, for example, of the invention may be cross reactive, that is, they may recognize a tau or an amyloid beta derived from a different source. Polyclonal antibodies have greater likelihood of cross reactivity. Alternatively, an antibody of the invention may be specific for a single form of tau or amyloid beta, such as the human amyloid beta proteins amyloid β₄₀ or amyloid β₄₂, or a fragment of a human amyloid beta protein.

Various procedures known in the art may be used for the production of polyclonal antibodies to a tau or an amyloid beta or derivatives, analogs or fragments thereof. For the production of an antibody, various host animals can be immunized by injection with the tau or amyloid beta, or a derivative (e.g., or fusion protein) thereof, including but not limited to rabbits, mice, rats, sheep, goats, etc. In one embodiment, tau or an amyloid beta or a fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward tau or amyloid beta, or analogs, derivatives or fragments thereof, any technique that provides for the production of antibody molecules by continuous cell lines in culture may be used. These include but are not limited to the hybridoma technique originally developed by Kohler and Milstein (Nature, 1975; 256:495-497), as well as the trioma technique, the human B-cell hybridoma technique (Kozbor et al., Immunology Today, 1983; 4:72; Cote et al., Proc. Natl. Acad. Sci. U.S.A., 1983; 80:2026-2030), and the EBV-hybridoma technique to produce human monoclonal antibodies (Cole et al., in Monoclonal Antibodies and Cancer Therapy, Alan R. Liss, Inc., 1985; pp. 77-96 (1985)). In an additional embodiment of the invention, monoclonal antibodies can be produced in germ-free animals utilizing recent technology (PCT/US90/02545). In fact, according to the invention, techniques developed for the production of “chimeric antibodies” (Morrison et al., J. Bacteriol., 1984; 159:870; Neuberger et al., Nature, 1984; 312:604-608; Takeda et al., Nature, 1985; 314:452-454) by splicing the genes from a mouse antibody molecule specific for amyloid beta together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies (Huston, U.S. Pat. Nos. 5,476,786 and 5,132,405 , and U.S. Pat. No. 4,946,778) can be adapted to produce e.g., tau or amyloid beta-specific single chain antibodies. An additional embodiment of the invention utilizes the techniques described for the construction of Fab expression libraries (Huse et al., Science, 1989; 246:1275-1281) to allow rapid and easy identification of monoclonal Fab fragments with the desired specificity for amyloid beta, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. Once the antibody is produced, the antibody may be employed in the assays noted above to screen bodily tissues or fluids for the presence of amyloid beta or fragments thereof. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of amyloid beta, one may assay generated hybridomas for a product which binds to the amyloid beta or fragment containing such epitope and choose those which do not cross-react with amyloid beta. For selection of an antibody specific to amyloid beta from a particular source, one can select on the basis of positive binding with amyloid beta expressed by or isolated from that specific source.

The foregoing antibodies can be used in methods known in the art relating to the localization and activity of the amyloid beta, e.g., for Western blotting, imaging tau or amyloid beta in situ, measuring levels thereof in appropriate physiological samples, etc. using any of the detection techniques mentioned herein or known in the art. The standard techniques known in the art for immunoassays are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, J. Clin. Chem. Clin. Biochem. 1984; 22:895-904.

In a specific embodiment, antibodies that agonize or antagonize the activity of tau or amyloid beta can be generated. Such antibodies can be tested using the assays described infra for identifying ligands.

One aspect of the invention provides a method of using an antibody against tau or an amyloid beta, for example Aβ₁₋₄₂, to diagnose a neurological disease in a subject. As tau or amyloid beta levels correlate with the presence of a neurological disease such as dementia, mild cognitive impairment or Alzheimer's Disease in a subject, with the presence of a neurological disease being determined by a psychiatric evaluation based on the presence of DSM-IV criteria and the presence of significant symptoms, tau or amyloid beta serve as a general biomarker for cognitive impairment and may be predictive of the future onset of a dementia such as Alzheimer's Disease. Alternatively, a tau or an amyloid beta may also serve as a marker for monitoring efficacy of therapy for a disorder, as described herein. Thus, the antibody compositions and methods provided herein are particularly deemed useful for the diagnosis of certain neurological diseases featuring cognitive impairment or dementia such as Alzheimer's Disease.

The diagnostic method of the invention provides contacting a cerebrospinal fluid sample isolated from a subject with an antibody which binds a tau or an amyloid beta (Ghiso et al. FEBS Letters 1997; 408:105-108). The antibody is allowed to bind to the antigen to form an antibody-antigen complex. The conditions and time required to form the antibody-antigen complex may vary and are dependent on the biological sample being tested and the method of detection being used. Once non-specific interactions are removed by, for example, washing the sample, the antibody-antigen complex is detected using any one of the immunoassays described above as well a number of well-known immunoassays used to detect and/or quantitate antigens (see, for example, Harlow and Lane, Antibodies: A Laboratory Manual, Cold Spring Harbor Laboratory, New York (1988) 555-612). Such well-known immunoassays include antibody capture assays, antigen capture assays, and two-antibody sandwich assays. In an antibody capture assay, the antigen is attached to solid support, and labeled antibody is allowed to bind. After washing, the assay is quantitated by measuring the amount of antibody retained on the solid support. In an antigen capture assay, the antibody is attached to a solid support, and labeled antigen is allowed to bind. The unbound proteins are removed by washing, and the assay is quantitated by measuring the amount of antigen that is bound. In a two-antibody sandwich assay, one antibody is bound to a solid support, and the antigen is allowed to bind to this first antibody. The assay is quantitated by measuring the amount of a labeled second antibody that binds to the antigen.

These immunoassays typically rely on labeled antigens, antibodies, or secondary reagents for detection. These proteins may be labeled with radioactive compounds, enzymes, biotin, or fluorochromes. Of these, radioactive labeling may be used for almost all types of assays. Enzyme-conjugated labels are particularly useful when radioactivity must be avoided or when quick results are needed. Biotin-coupled reagents usually are detected with labeled streptavidin. Streptavidin binds tightly and quickly to biotin and may be labeled with radioisotopes or enzymes. Fluorochromes, although requiring expensive equipment for their use, provide a very sensitive method of detection. Those of ordinary skill in the art will know of other suitable labels which may be employed in accordance with the present invention. The binding of these labels to antibodies or fragments thereof may be accomplished using standard techniques such as those described by Kennedy et al., Clin. Chim. Acta 1976; 70: 1-31), and Schurs et al. Clin. Chim Acta 1977; 81: 1-40).

In accordance with the diagnostic method of the invention, the presence or absence of the antibody-antigen complex is correlated with the presence or absence in the biological sample, e.g. cerebrospinal fluid, of the antigen, or a peptide fragment thereof. A biological sample containing elevated levels of said antigen (a tau or an amyloid beta) is indicative of a neurological disease such as mild cognitive impairment or Alzheimer's Disease in a subject from which the biological sample was obtained. Accordingly, the diagnostic method of the invention may be used as part of a routine screen in subjects suspected of having or being at risk for developing a cognitive impairment, a dementia or Alzheimer's Disease. Moreover, the diagnostic method of the invention may be used alone or in combination with other well-known diagnostic methods to confirm the presence of a neurological disease.

The diagnostic method of the invention further provides that an antibody of the invention may be used to monitor the levels of a tau or an amyloid beta antigen in patient samples at various intervals of drug treatment to identify whether and to which degree the drug treatment is effective in reducing or inhibiting the symptoms associated with the neurological disease, such reduction being an indication that the therapy may ultimately result in amelioration and/or cure of the disease. Furthermore, antigen levels may be monitored using an antibody of the invention in studies evaluating efficacy of drug candidates in model systems and in clinical trials. The antigens provide for surrogate biomarkers in biological fluids to non-invasively assess the global status of the disease. For example, using an antibody of this invention, antigen levels may be monitored in biological samples of individuals treated with known or unknown therapeutic agents or toxins. This may be accomplished with cell lines in vitro or in model systems and clinical trials, depending on the disease. Persistently increased total levels of tau or amyloid beta antigen in biological samples during or immediately after treatment with a drug candidate indicates that the drug candidate has little or no effect. Likewise, the reduction in total levels of tau or amyloid beta antigen indicates that the drug candidate is effective in reducing or inhibiting the symptoms of the disease. Furthermore, the continued reduction of tau or amyloid beta in the subject may ultimately result in full remission of the individual suffering from the disease. This may provide valuable information at all stages of pre-clinical drug development, clinical drug trials as well as subsequent monitoring of patients undergoing drug treatment.

### Antibody Labels

The tau or amyloid beta proteins of the present invention, antibodies to tau or amyloid beta proteins, and nucleic acids that hybridize to tau or amyloid beta genes (e.g. probes) etc. can all be labeled. Suitable labels include enzymes, fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the receptor and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. Such labels may also be appropriate for the nucleic acid probes used in binding studies with tau or amyloid beta. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. ultraviolet light to promote fluorescence. Among examples of colored labels, which can be used according to the present invention, include metallic sol particles, for example, gold sol particles such as those described by Leuvering, U.S. Pat. No. 4,313,734; dye sole particles such as described by Gribnau et al., U.S. Pat. No. 4,373,932 and May et al., WO 88/08534; dyed latex such as described by May, supra, Snyder, EP-A 0 280 559 and 0 281 327; or dyes encapsulated in liposomes as described by Campbell et al., U.S. Pat. No. 4,703,017. Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 1980; 70:419-439 and in U.S. Pat. No. 4,857,453.

Suitable enzymes include, but are not limited to, alkaline phosphatase and horseradish peroxidase.

In addition, tau or amyloid beta, or a fragment thereof can be modified to contain a marker protein such as green fluorescent protein as described in U.S. Pat. No. 5,625,048 filed, WO 97/26333, and WO 99/64592 all of which are hereby incorporated by reference in their entireties. Other labels for use in the invention include magnetic beads or magnetic resonance imaging labels. In another embodiment, a phosphorylation site can be created on an antibody of the invention for labeling with ³²P, e.g., as described by Pestka, European Patent No. 0372707 or Foxwell et al., U.S. Pat. No. 5,459,240.

As exemplified herein, proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions). Quantification of amyloid beta can also be done using staining with Congo red with subsequent image analysis (Kindy et al., Am. J. Pathol. 1998; 152: 1387-1395; Kisilevsky et al., Nat. Med. 1995; 1: 143-148).

### Other Diagnostic Means for Determining Levels of Tau or Amyloid Beta

### Cell-Based Reporters and Instrumentation

Cellular screening techniques can be broadly classified into two groups: semi-biochemical approaches that involve the analysis of cell lysates, or live cell assays. Whole cell assay methodologies vary with respect to assay principle, but have largely in common a form of luminescence or fluorescence for detection. Luminescence is a phenomenon in which energy is specifically channeled to a molecule to produce an excited state. Luminescence includes fluorescence, phosphorescence, chemiluminescence and bioluminescence.

An ever-increasing list of fluorescent proteins includes the widely-used GFP derived from Aequorea Victoria and spectral variants thereof. The list includes a variety of fluorescent proteins derived from other marine organisms; bacteria; fungi; algae; dinoflagellates; and certain terrestrial species. These reporters have the advantage of not requiring any exogenous substrates or co-factors for the generation of a signal but do require an external source of radiation for excitation of the intrinsic fluorophore. In addition, the increasing availability of genes encoding a broad spectrum of fluorescent reporter proteins enables the construction of assays tailored for specific applications, cell types, and detection systems.

Different classes of luminescent proteins, luciferases, have been discovered in bacteria and eukaryotes. Luciferases are proteins that catalyze the conversion of a natural substrate into a product that emits light in the visible spectrum and thus require no external radiation source. Monomeric forms of luciferase have been cloned from firefly, Renilla, and other organisms. Firefly luciferase is the most common of the bioluminescent reporters and is a 61 kDa monomeric enzyme that catalyzes a two-step oxidation reaction to yield light. Renilla luciferase is a 31 kDa monomeric enzyme that catalyzes the oxidation of coelenterazine to yield coelenteramide and blue light of 480 nm. Substrates for luciferase are widely available from commercial suppliers such as Promega Corporation and Invitrogen Molecular Probes.

A variety of useful enzymatic reporters are enzymes that either generate a fluorescent signal or are capable of binding small molecules that can be tagged with a fluorescent moiety to serve as a fluorescent probe. For example, dihydrofolate reductase (DHFR) is capable of binding methotrexate with high affinity; a methotrexate-fluorophore conjugate can serve as a quantitative fluorescent reagent for the measurement of the amount of DHFR within a cell. By tagging methotrexate with any of a number of fluorescent molecules such as fluorescein, rhodamine, Texas Red, BODIPY and other commercially available molecules (such as those available from Molecular Probes/Invitrogen and other suppliers) a range variety of fluorescent readouts can be generated. The wide range of techniques of immunohistochemistry and immunocytochemistry can be applied to whole cells. For example, ligands and other probes can be tagged directly with fluorescein or another fluorophore for detection of binding to cellular proteins; or can be tagged with enzymes such as alkaline phosphatase or horseradish peroxidase to enable indirect detection and localization of signal.

Many other enzymes can be used to generate a fluorescent signal in live cells by using specific, cell-permeable substrate that either becomes fluorescent or shifts its fluorescence spectrum upon enzymatic cleavage. For example, substrates for β-lactamase exist whose fluorescence emission properties change in a measurable way upon cleavage of a β-lactam core moiety to which fluorophores are attached. Changes include, shifts in fluorophore absorption or emission wavelengths, or cleavage of a covalent assembly of emission-absorption-matched fluorophore pairs that in the covalently-assembled form sustain resonance energy transfer between the two fluorophores that is lost when the two are separated. Membrane-permeant, fluorescent BLA substrates such as the widely-used CCF2/AM allow the measurement of gene expression in live mammalian cells in the absence or presence of compounds from a biologically active chemical library.

Luminescent, fluorescent or bioluminescent signals are easily detected and quantified with any one of a variety of automated and/or high-throughput instrumentation systems including fluorescence multi-well plate readers, fluorescence activated cell sorters (FACS) and automated cell-based imaging systems that provide spatial resolution of the signal. A variety of instrumentation systems have been developed to automate HCS including the automated fluorescence imaging and automated microscopy systems developed by Cellomics, Amersham, TTP, Q3DM, Evotec, Universal Imaging and Zeiss. Fluorescence recovery after photobleaching (FRAP) and time lapse fluorescence microscopy have also been used to study protein mobility in living cells. Although the optical instrumentation and hardware have advanced to the point that any bioluminescent signal can be detected with high sensitivity and high throughput, the existing assay choices are limited either with respect to their range of application, format, biological relevance, or ease of use.

### Transcriptional Reporter Assays

Cell-based reporters are often used to construct transcriptional reporter assays, which allow monitoring of the cellular events associated with signal transduction and gene expression. Reporter gene assays couple the biological activity of a target to the expression of a readily detected enzyme or protein reporter. Based upon the fusion of transcriptional control elements to a variety of reporter genes, these systems “report” the effects of a cascade of signaling events on gene expression inside cells. Synthetic repeats of a particular response element can be inserted upstream of the reporter gene to regulate its expression in response to signaling molecules generated by activation of a specific pathway in a live cell. The variety of transcriptional reporter genes and their application is very broad and includes drug screening systems based on β-galactosidase (β-gal), luciferase, alkaline phosphatase (luminescent assay), GFP, aequorin, and a variety of newer bioluminescent or fluorescent reporters.

In general, transcription reporter assays have the capacity to provide information on the response of a pathway to natural or synthetic chemical agents on one or more biochemical pathways, however they only indirectly measure the effect of an agent on a pathway by measuring the consequence of pathway activation or inhibition, and not the site of action of the compound. For this reason, mammalian cell-based methods have been sought to directly quantitate protein-protein interactions that comprise the functional elements of cellular biochemical pathways and to develop assays for drug discovery based on these pathways.

Cellular Assays for Individual Proteins Tagged with Fluorophores or Luminophores.

Subcellular compartmentalization of signaling proteins is an important phenomenon not only in defining how a biochemical pathway is activated but also in influencing the desired physiological consequence of pathway activation. This aspect of drug discovery has seen a major advance as a result of the cloning and availability of a variety of intrinsically fluorescent proteins with distinct molecular properties.

High-content (also known as high-context) screening (HCS) is a live cell assay approach that relies upon image-based analysis of cells to detect the subcellular location and redistribution of proteins in response to stimuli or inhibitors of cellular processes. Fluorescent probes can be used in HCS; for example, receptor internalization can be measured using a fluorescently-labeled ligand that binds to the transferrin receptor. Often, individual proteins are either expressed as fusion proteins, where the protein of interest is fused to a detectable moiety such as GFP, or are detected by immunocytochemistry after fixation, such as by the use of an antibody conjugated to Cy3 or another suitable dye. In this way, the subcellular location of a protein can be imaged and tracked in real time. One of the largest areas of development is in applications of GFP color-shifted mutants and other more recently isolated new fluorescent proteins, which allow the development of increasingly advanced live cell assays such as multi-color assays. A range of GFP assays have been developed to analyze key intracellular signaling pathways by following the redistribution of GFP fusion proteins in live cells. For drug screening by HCS the objective is to identify therapeutic compounds that block disease pathways by inhibiting the movement of key signaling proteins to their site of action within the cell.

Tagging a protein with a fluorophore or a luminophore enables tracking of that particular protein in response to cell stimuli or inhibitors. For example, the activation of cell signaling by TNF can be detected by expressing the p65 subunit of the NFkB transcription complex as a GFP fusion and then following the redistribution of fluorescence from the cytosolic compartment to the nuclear compartment of the cell within minutes after TNF stimulation of live cells (Schmid et al., J. Biol. Chem. 2000; 275: 17035-17042). What has been unique about these approaches is the ability to allow monitoring of the dynamics of individual protein movements in living cells, thus addressing both the spatial and temporal aspects of signaling.

It should be understood by persons of ordinary skill in the an that various modifications may be made to the presently claimed invention that would lie within the spirit of the invention and the scope of the appended claims. For example, biomarkers other then the Tau and amyloid beta peptides can be tested for in the CSF. There are many brain derived substances that can be found in CSF, and the claimed invention is a useful way to properly calculate their concentration.

While the diagnosis of and study of Alzheimer's Disease is of particular interest, many other types of brain impairment/disease can be tested for. The present invention provides, inter alia, a valuable diagnostic tool to check the progression of persons as time passes. The scans from the MRI can be stored in a database, that can be attached locally, over the Internet, on a writable CD, electronically stored, etc, as desired. Furthermore, the present invention also contemplates a system for testing, the system can be a unix workstation, personal computer, midframe computer, etc. The algorithm, which can be part of the MIDAS program, can also be adapted to run on any operating system to fit need. The type of MRI machines used are also according to need.

Finally, it should also be understood that often psychometric/psychiatric and neurologic evaluations are made in conjunction with the corrected biomarker factor to increase the accuracy of the diagnosis. The algorithm may provide a factor that can be multiplied against the initial CSF biomarker concentration level, or the algorithm may alternatively provide the corrected score. Either provision of the correction factor and/or the score is clearly within the spirit of the invention and the scope of the appended claims.

### DETAILED DESCRIPTION OF THE PREFERRED EMBODIMENTS

### EXAMPLE 1

### Correcting the CSF P-tau₂₃₁ Level for the Ventricular Volume

### Materials and Methods

In a longitudinal MRI and CSF study, in a one year study of an eleven person NL control group (X_{gds}=1.6, plus or minus 0.5, Xₘₘₛₑ=29.4, plus or minus 0.7) and eight mildly cognitively impaired (MCI) patients (X_{gds}=3, Xₘₘₛₑ=28.5, plus or minus 1.2) the cross-sectional and longitudinal hippocampal and CSF volumes, and from the lumbar puncture, the CSF levels (pg/ml) of P-tau₂₃₁, Amyloid beta (AB) 1-40 (40) and Aβ₄₂. The groups did not differ in age (range 52-81 years) for any of the measures.

At baseline, follow-up, and longitudinally, the MCI group was compared with the NL control group. During the study, one NL subject converted to MCI and two MCI subjects converted to Alzheimer's Disease.

### Results

The hippocampal volume was significantly reduced in the MCI group at both baseline (15%; X_{NL}=33+/−35, X_{MCI}2.8+/−0.26, p<0.01) and follow-up (19%; X_{NL}=3.2+/−0.24, X_{MCI}=2.6+/−0.30, p<0.001). The longitudinal hippocampal results did not reach significance. The MRI derived CSF volume did not show any cross-sectional or longitudinal changes. Significant elevations in P-tau₂₃₁ levels were found in MCI relative to controls, at both baseline (250%; X_{NL}=152.7+/−0.182.3, X_{MC}=534.7+/−451.8; t (8.7)=−2.3, p<.ltoreq.0.05), and follow-up (710%; X_{NL}=69.3+/−48.5, X_{MCI}=561.5+/−447.2; t (8.2)=−3.3, p=0.01).

In addition, Aβ₄₀ was also increased at baseline (32%; X_{NL}=9,396.4+/−2,295.7, X_{MCI}=12,393.1+/−2,388.5; (t (17)=−2.8, p<0.05) and follow-up (36%; X_{NL}=8,564.6+/−2,174.2, X_{MCI}=11,643.4+/−2,809.1; (t (17)=−2.7, p<0.05). No longitudinal effects were observed for the CSF protein levels. The analyses were repeated using a logarithmic transformation of the data and observed similar results as the aforementioned. The highest baseline and follow-up patient classification accuracies (sensitivities=88% and specificities>90%, p<0.01) were found with the combination of P-tau₂₃₁, Aβ₄₀, and hippocampal volume. There was also a significant relationship between the hippocampal volumes and P-tau₂₃₁ level at baseline (r=−0.48, p<0.05) and at follow up (r=−0.59, p<0.01).

Correcting the CSF P-tau₂₃₁ level for the ventricular volume produces the only significant longitudinal effect ((t)17)=−2.6, p<0.05). The longitudinal increase in the corrected P-tau₂₃₁ data correctly classified 74% of the total sample with an odds ratio=2.1, (p<0.01). The foregoing data and findings are set forth in de Leon et al., Neuroscience Letters, 2002; 333:183-186, and this reference is incorporated herein by reference in its entirety.

### EXAMPLE 2

### Longitudinal CSF Tau Dilution in Preclinical Alzheimer's disease

### Materials and Methods

A New York University Institutional Review Board approved longitudinal study of 26 cognitively normal subjects (NL) with an age range of 61 to 86 was conducted. The subjects received minimum of three annual follow-up clinical and neuropsychological evaluations for up to 10 years. (See, Table 1)

TABLE 1
Sample characteristics

| | NL-Stable | | NL-Decline | |
|---|---|---|---|---|
| | (n = 19) | | (n = 7) | |
| Age, baseline (range and mean years) | 61-85, | | 69-86, | |
| | 68.5 (7.2) | | 75.1 (6.3) * | |
| Gender, M/F | 9/10 | | 3/4 | |
| Education (years) | 16.7 | (2.1) | 16 | (3.2) |
| MMSE Baseline | 29.5 | (.77) | 29.3 | (.51) |
| MMSE Follow-up | 29.3 | (.82) | 29.1 | (.69) |
| PARD Baseline | 9.5 | (2.6) | 7.9 | (2.1) |
| PARD Follow-up | 9.5 | (3.3) | 6.4 | (3.3) |
| ApoE ε4 (subject frequency) | .21 | | .28 | |
| Time to CSF follow up (years from | 2.87 | (.81) | 3.19 | (1.11) |
| baseline) | | | | |
| Last clinical visit (Years from baseline) | 6 | (2.2) | 6 | (2.2) |

Values are mean (SD) or percentage not correct as some are frequency and others are fractions.
* Different from stable NL, p ≦ 0.05
PARD: Delayed recall of the NYU paragraphs test

CSF T-tau and volumetric MRI were obtained at the baseline and 3±1 years after baseline. At baseline, all subjects were normal (NL) defined as having a Clinical Dementia Rating (CDR) (Morris Neurology, 1993; 43(11): 2412-2414) score of CDR=0, a Global Deterioration Scale (GDS) (Reisberg et al., Psychopharmacol Bull, 1988; 24(4): 661-663) score of GDS=1 or 2, Mini-mental status (MMSE) (Folstein et al., Arch Gen Psychiatry, 1983; 40(7): 812)>28. The delayed paragraph recall from a neuropsychological test battery with longitudinal norms (De Santi et al., Neuropsychology, 2008; 22(4): 469-484) was used to assess clinical progression. At each examination, subjects were evaluated for clinical change to mild cognitive impairment (MCI) or Alzheimer's Disease (AD). The diagnosis of decline to MCI was based on the following criteria: progressive cognitive complaints (typically memory), increased complaints corroborated by an informant, CDR=0.5, GDS=3 and clinically recognizable cognitive impairment without fulfilling either the DSM-IV (American Psychiatric Association. Diagnostic and statistical manual of mental disorders, fourth edition (DSM-IV). Washington, D.C. ed.: American Psychiatric Association, 1994) or NINCDS-ADRDA (McKhann, et al., Neurology, 1984; 34(7): 939-944) criteria for dementia or AD. Patients transitioning to Alzheimer's Disease (AD) fulfilled the DSM-IV criteria for dementia and the NINCDS-ADRDA criteria for AD, and had GDS scores ≧4 and CDR≧1.0. Subjects remaining within normal (NL) limits are referred to as NL-Stable and those showing progressive cognitive change at clinical follow-up as NL-Decline. Subjects with neurological abnormalities (e.g. cortical stroke, brain tumor, head trauma), or significant psychiatric disorders, or other conditions deemed to influence cognitive performance were excluded.

### Cognitive Outcome Measures

The delayed paragraph recall subtest (PARD) from the Guild Memory Test (Gilbert et al., Percept Mot Skills, 1968; 27(1): 277-278), also known as the NYU Paragraph Recall Test was used to examine memory performance between groups, and in association with Tau measures, at baseline and longitudinally. For both the baseline and the follow-up, scores were converted into a standardized score (z-score) based on a normative reference group, accounting for age, education and gender (De Santi et al., Neuropsychology, 2008; 22(4): 469-484). PARD has been routinely administered at NYU since 1975. Previously we observed this measure to be the single best cognitive predictor of decline to MCI or AD among in non-demented individuals (Kluger et al., J Geriatr Psychiatry Neurol, 1999; 12(4): 168-179).

### MRI Acquisition

MR Images were acquired on a research dedicated 1.5T GE Signa Imager scanner (General Electric, Milwaukee, USA). All subjects received diagnostic and research MRI studies. The diagnostic study was used to satisfy the exclusion criteria and included 2 mm coronal T2-weighted and contiguous 3 mm Fluid attenuated Inversion-Recovery (FLAIR) fast spin echo axial images. The research study was a high-resolution T1-weighted 3D fast gradient-echo acquired in coronal (TR: 35 ms, TE: 9 ms, FA: 60, FOV: 18×18 cm, matrix: 256×192, with a slice thickness of 1.6 mm.

### MRI Image Analysis

Baseline and follow-up MR images were transferred offline to a Linux workstation for post processing. FreeSurfer (version 5.1, http://surfer.nmr.mgh.harvard.edu) with its longitudinal processing pipeline (http://surfer.nmr.mgh.harvard.edu/fswiki/) was implemented and used to segment the ventricle and intracranial volumes. Ventricle CSF volume (V-CSF) was estimated using the sum of the lateral ventricle and third ventricles. The intracranial CSF volume (IC-CSF) was segmented by SPM8 with a whole brain mask (http://www.fil.ion.ucl.ac.uk/spm/doc/books/hbf2/pdfs/Ch5.pdf). All images were examined in a quality control step to ensure the precision of the volumetric solutions.

### Lumbar Puncture, CSF Collection and Assays

Fifteen milliliters of clear CSF was collected at 11 am into three polypropylene tubes using a 25G LP needle guided by fluoroscopy. CSF samples were kept on ice for a maximum of 1 h before being centrifuged for 10 min at 1500×g at 4° C. Samples were frozen and stored in 250 uL polypropylene tubes at −80° C. until defrosted for this project. CSF T-tau was determined using the commercially available INNOTEST hTAU Antigen kit from Inno-genetics (Blennow et al., Mol Chem Neuropathol, 1995; 26(3): 231-245). For T-tau, the detection limit is 60 pg/ml and the coefficients of variability are 5.5% (intra-assay) and 11.6% (inter-assay).

T-au Load measurements: The T-tau load (ng) in the CSF was estimated in two ways, by multiplying the T-tau concentration (pg/ml) by the CSF volume (either by the V-CSF (ml) or the IC-CSF (ml)) and dividing by 1000 (de Leon et al., Neurosci Lett. 2002; 333(3): 183-186).

### Statistical Analysis

Demographic differences between the declining and non-declining groups for the continuous variables (e.g. age) were examined with t-tests; and, for categorical variables (e.g. gender), group differences were evaluated using chi-squared analyses. Age was the only demographic variable that significantly differed between diagnostic groups was included as a covariate in all analyses. Baseline, follow-up, and longitudinal differences between the outcome were examined with analysis of covariance (ANCOVA) with baseline age as a covariate.

Annual Change Rate (ACR) was calculated by the formula (Longitudinally measured change/baseline measurement/follow-up duration×100). Pearson correlation coefficient (r) and nonparametric Spearman correlations were used to examine the associations between delayed memory (PARD) and brain and CSF measurements. There were no violations in model assumptions of normality, equality of variances, or independence. All analyses were performed with SPSS.19, Chicago, Ill., with p values declared statistically significant when p<0.05.

### Results

### Demographic Characteristics

19 of 26 of subjects (73%) remained NL at all clinical follow ups out to an average of 6 years (range 3-9 years). Seven subjects (27%) declined to MCI within 2-7 years of the baseline exam, and three received an AD diagnosis on average 6 years after baseline (Table 1). There were no gender, education, baseline MMSE scores, observation interval or ApoE ε4 allele frequency differences between NL-Stable and NL-Decline groups. (See, Table 1) The NL-Decliners were older than NL-Stable. Age was used as a covariate in all of the models.

### Cross-Sectional MRI and Tau Measures

There were no cross-sectional differences between NL-Decline and NL-Stable groups for the CSF volume measurements, the T-tau level, or the T-tau load.

### Longitudinal Tau and MRI Measures

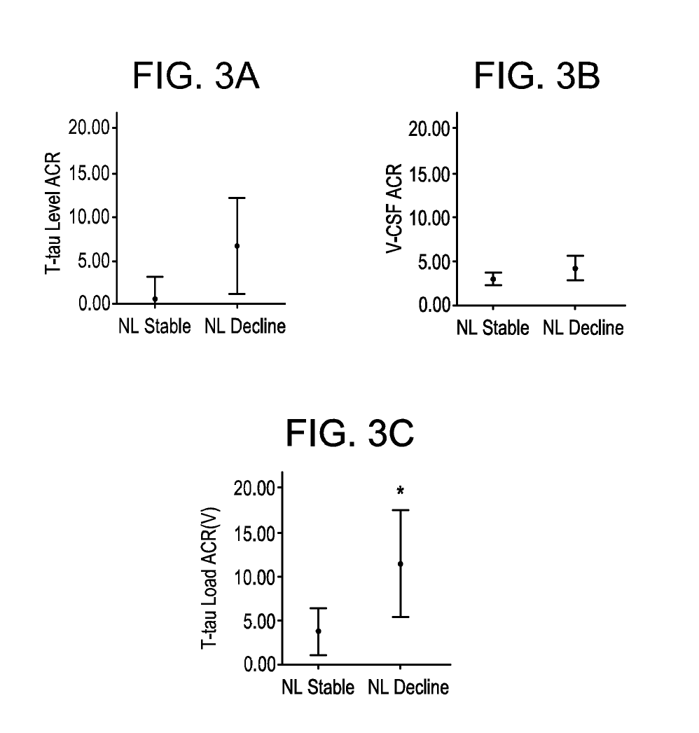

Neither the rate of change in the T-tau level nor the rate of change in the CSF volume measurements reached significance (Table 2). However, the rate of change in the T-tau load (V) and the T-tau load (ICV) were significantly different between NL-Decline and NL-Stable groups (F (2, 23) =6.15 p=0.02) and (F(2.23)=5.79 p=0.03, respectively. (See, FIGS. 3a and b)

TABLE 2
Cross-sectional and Longitudinal MRI and CSF Measurements

| | NL-Stable | NL-Decline | |
|---|---|---|---|
| | (n = 19) | (n = 7) | P value |
| MRI Measurements | | | |
| V-CSF (ml)Baseline | 31 (11) | 51 (23) | 0.26 |
| V-CSF (ml)Follow-up | 33 (12) | 57 (26) | 0.12 |
| IC-CSF (ml) Baseline | 246 (46) | 320 (90) | 0.35 |
| IC-CSF (ml) Follow-up | 257 (48) | 350 (96) | 0.11 |
| V-ACR | 2.9 (1.4) | 4.2 (1.5) | 0.15 |
| IC-ACR | 1.9 (1.3) | 3.0 (1.4) | 0.22 |
| Tau Level Measurements | | | |
| T-tau Level (pg/ml) Baseline | 290 (89) | 342 (124) | 0.71 |
| T-tau Level (pg/ml) Follow-up | 293 (90) | 400 (164) | 0.22 |
| T-tau Level ACR | 0.6 (5.3) | 6.6 (5.9) | 0.06 |
| Tau Load Measurements | | | |
| T-tau Load(IC) (ng) Baseline | 71 (23) | 116 (72) | 0.52 |
| T-tau Load(IC) (ng) Follow-up | 75 (25) | 151 (104) | 0.18 |
| T-tau Load(V) (ng) Baseline | 31 (11) | 51 (23) | 0.28 |
| T-tau Load(V) (ng) Follow-up | 34 (12) | 58 (26) | 0.13 |
| T-tau Load V-ACR | 3.69 (5.5) | 11.5 (6.5) | 0.02* |
| T-tau Load IC-ACR | 2.62 (5.3) | 10.11 (6.3) | 0.03* |

Values are mean (SD),
*p ≦ 0.05
ACR: Annual Change Rate; T-tau Load(V): T-tau load calculated by ventricle CSF volume; T-tau Load(IC); T-tau load calculated by IC-CSF volume.

### Correlations Between Memory Test and Tau Measures

There were no significant baseline correlations between T-tau measures and PARD. Both T-tau level and T-tau load were significantly correlated to PARD Z-scores at follow-up (T-tau level r=−0.42, p=0.04; T-tau load(V) r=−0.41, p=0.04; T-tau load(IC) r=−0.48 p=0.02). Longitudinally, the PARD Z-score ACR was negatively correlated with both the ACR for the T-tau Load(V) r=−0.42, p=0.04) and T-tau Load(IC)r=−0.44, p=0.03). No longitudinal effects were found for the Tau level (p>0.05).

These data demonstrate that longitudinal T-tau load is useful biomarker for the presymptomatic detection of brain damage as related to AD.

| SEQ ID NO: 1 is the nucleic acid sequence encoding human amyloid beta | |
|---|---|
| precursor protein | |
| gctgactcgc ctggctctga gccccgccgc cgcgctcggg ctccgtcagt ttcctcggca | 60 |
| gcggtaggcg agagcacgcg gaggagcgtg cgcgggggcc ccgggagacg gcggcggtgg | 120 |
| cggcgcgggc agagcaagga cgcggcggat cccactcgca cagcagcgca ctcggtgccc | 180 |
| cgcgcagggt cgcgatgctg cccggtttgg cactgctcct gctggccgcc tggacggctc | 240 |
| gggcgctgga ggtacccact gatggtaatg ctggcctgct ggctgaaccc cagattgcca | 300 |
| tgttctgtgg cagactgaac atgcacatga atgtccagaa tgggaagtgg gattcagatc | 360 |
| catcagggac caaaacctgc attgatacca aggaaggcat cctgcagtat tgccaagaag | 420 |
| tctaccctga actgcagatc accaatgtgg tagaagccaa ccaaccagtg accatccaga | 480 |
| actggtgcaa gcggggccgc aagcagtgca agacccatcc ccactttgtg attccctacc | 540 |
| gctgcttagt tggtgagttt gtaagtgatg cccttctcgt tcctgacaag tgcaaattct | 600 |
| tacaccagga gaggatggat gtttgcgaaa ctcatcttca ctggcacacc gtcgccaaag | 660 |
| agacatgcag tgagaagagt accaacttgc atgactacgg catgttgctg ccctgcggaa | 720 |
| ttgacaagtt ccgaggggta gagtttgtgt gttgcccact ggctgaagaa agtgacaatg | 780 |
| tggattctgc tgatgcggag gaggatgact cggatgtctg gtggggcgga gcagacacag | 840 |
| actatgcaga tgggagtgaa gacaaagtag tagaagtagc agaggaggaa gaagtggctg | 900 |
| aggtggaaga agaagaagcc gatgatgacg aggacgatga ggatggtgat gaggtagagg | 960 |
| aagaggctga ggaaccctac gaagaagcca cagagagaac caccagcatt gccaccacca | 1020 |
| ccaccaccac cacagagtct gtggaagagg tggttcgagt tcctacaaca gcagccagta | 1080 |
| cccctgatgc cgttgacaag tatctcgaga cacctgggga tgagaatgaa catgcccatt | 1140 |
| tccagaaagc caaagagagg cttgaggcca agcaccgaga gagaatgtcc caggtcatga | 1200 |
| gagaatggga agaggcagaa cgtcaagcaa agaacttgcc taaagctgat aagaaggcag | 1260 |
| ttatccagca tttccaggag aaagtggaat ctttggaaca ggaagcagcc aacgagagac | 1320 |
| agcagctggt ggagacacac atggccagag tggaagccat gctcaatgac cgccgccgcc | 1380 |
| tggccctgga gaactacatc accgctctgc aggctgttcc tcctcggcct cgtcacgtgt | 1440 |
| tcaatatgct aaagaagtat gtccgcgcag aacagaagga cagacagcac accctaaagc | 1500 |
| atttcgagca tgtgcgcatg gtggatccca agaaagccgc tcagatccgg tcccaggtta | 1560 |
| tgacacacct ccgtgtgatt tatgagcgca tgaatcagtc tctctccctg ctctacaacg | 1620 |
| tgcctgcagt ggccgaggag attcaggatg aagttgatga gctgcttcag aaagagcaaa | 1680 |
| actattcaga tgacgtcttg gccaacatga ttagtgaacc aaggatcagt tacggaaacg | 1740 |
| atgctctcat gccatctttg accgaaacga aaaccaccgt ggagctcctt cccgtgaatg | 1800 |
| gagagttcag cctggacgat ctccagccgt ggcattcttt tggggctgac tctgtgccag | 1860 |
| ccaacacaga aaacgaagtt gagcctgttg atgcccgccc tgctgccgac cgaggactga | 1920 |
| ccactcgacc aggttctggg ttgacaaata tcaagacgga ggagatctct gaagtgaaga | 1980 |
| tggatgcaga attccgacat gactcaggat atgaagttca tcatcaaaaa ttggtgttct | 2040 |
| ttgcagaaga tgtgggttca aacaaaggtg caatcattgg actcatggtg ggcggtgttg | 2100 |
| tcatagcgac agtgatcgtc atcaccttgg tgatgctgaa gaagaaacag tacacatcca | 2160 |
| ttcatcatgg tgtggtggag gttgacgccg ctgtcacccc agaggagcgc cacctgtcca | 2220 |
| agatgcagca gaacggctac gaaaatccaa cctacaagtt ctttgagcag atgcagaact | 2280 |
| agacccccgc cacagcagcc tctgaagttg gacagcaaaa ccattgcttc actacccatc | 2340 |
| ggtgtccatt tatagaataa tgtgggaaga aacaaacccg ttttatgatt tactcattat | 2400 |
| cgccttttga cagctgtgct gtaacacaag tagatgcctg aacttgaatt aatccacaca | 2460 |
| tcagtaatgt attctatctc tctttacatt ttggtctcta tactacatta ttaatgggtt | 2520 |
| ttgtgtactg taaagaattt agctgtatca aactagtgca tgaatagatt ctctcctgat | 2580 |
| tatttatcac atagcccctt agccagttgt atattattct tgtggtttgt gacccaatta | 2640 |
| agtcctactt tacatatgct ttaagaatcg atgggggatg cttcatgtga acgtgggagt | 2700 |
| tcagctgctt ctcttgccta agtattcctt tcctgatcac tatgcatttt aaagttaaac | 2760 |
| atttttaagt atttcagatg ctttagagag attttttttc catgactgca ttttactgta | 2820 |
| cagattgctg cttctgctat atttgtgata taggaattaa gaggatacac acgtttgttt | 2880 |
| cttcgtgcct gttttatgtg cacacattag gcattgagac ttcaagcttt tctttttttg | 2940 |
| tccacgtatc tttgggtctt tgataaagaa aagaatccct gttcattgta agcactttta | 3000 |
| cggggcgggt ggggaggggt gctctgctgg tcttcaatta ccaagaattc tccaaaacaa | 3060 |
| ttttctgcag gatgattgta cagaatcatt gcttatgaca tgatcgcttt ctacactgta | 3120 |
| ttacataaat aaattaaata aaataacccc gggcaagact tttctttgaa ggatgactac | 3180 |
| agacattaaa taatcgaagt aattttgggt ggggagaaga ggcagattca attttcttta | 3240 |
| accagtctga agtttcattt atgatacaaa agaagatgaa aatggaagtg gcaatataag | 3300 |
| gggatgagga aggcatgcct ggacaaaccc ttcttttaag atgtgtcttc aatttgtata | 3360 |
| aaatggtgtt ttcatgtaaa taaatacatt cttggaggag caaaaaaaaa aaaaaa | 3416 |
| SEQ ID NO: 2 is the nucleic acid encoding human Aβ₄₀ peptide | |
| gatgcagaat tccgacatga ctcaggatat gaagttcatc atcaaaaatt ggtgttcttt | 60 |
| gcagaagatg tgggttcaaa caaaggtgca atcattggac tcatggtggg cggtgttgtc | 120 |
| SEQ ID NO: 3 is the amino acid sequence of human Aβ₄₀ peptide | |
| Asp Ala Glu Phe Arg His Asp Ser Gly Tyr Glu Val His His Gln Lys | |
| Leu Val Phe Phe Ala Glu Asp Val Gly Ser Asn Lys Gly Ala Ile Ile | |
| Gly Leu Met Val Gly Gly Val Val | |
| SEQ ID NO: 4 is the nucleic acid encoding human Aβ₄₂ | |
| gatgcagaat tccgacatga ctcaggatat gaagttcatc atcaaaaatt ggtgttcttt | 60 |
| gcagaagatg tgggttcaaa caaaggtgca atcattggac tcatggtggg cggtgttgtc | 120 |
| atagcg | 126 |
| SEQ ID NO: 5 is the amino acid sequence of human Aβ₄₂ peptide | |
| Asp Ala Glu Phe Arg His Asp Ser Gly Tyr Glu Val His His Gln Lys | |
| Leu Val Phe Phe Ala Glu Asp Val Gly Ser Asn Lys Gly Ala Ile Ile | |
| Gly Leu Met Val Gly Gly Val Val Ile Ala | |

We claim:

1. A method for diagnosing a neurological disease in a subject comprising
(a) obtaining a cerebrospinal fluid (CSF) sample from a subject;
(b) providing a cerebrospinal fluid (CSF) correction factor for CSF obtained from a subject for at least one biomarker; and
(c) determining whether the biomarker is present in elevated amounts or concentrations in the cerebrospinal fluid (CSF) sample.

2. A method according to claim 1 wherein the neurological disease is selected from the group consisting of impaired cognition and dementia.

3. A method according to claim 1 wherein the neurological disease is selected from the group consisting of Alzheimer's Disease (AD) and Mild Cognitive Impairment (MCI).

4. A method according to claim 1 wherein the biomarker is selected from the group consisting of a tau protein and an amyloid beta (Aβ).

5. A method according to claim 1 wherein the biomarker is measured by a quantitative method selected from the group consisting of an immunological or biochemical assay specific for the biomarker, an enzyme-linked immunosorbent assay (ELISA), a Western blot assay, a Northern blot assay, and a Southern blot assay.

6. A method for screening for or assessing the risk of developing a neurological disease in a subject comprising
(a) obtaining a cerebrospinal fluid (CSF) sample from a subject;
(b) providing a cerebrospinal fluid (CSF) correction factor for CSF obtained from a subject for at least one biomarker; and
(c) determining whether the biomarker is present in elevated amounts or concentrations in the cerebrospinal fluid (CSF) sample.

7. A method according to claim 6 wherein the neurological disease is selected from the group consisting of impaired cognition and dementia.

8. A method according to claim 6 wherein the neurological disease is selected from the group consisting of Alzheimer's Disease (AD) and Mild Cognitive Impairment (MCI).

9. A method according to claim 6 wherein the biomarker is selected from the group consisting of a tau protein and an amyloid beta (Aβ).

10. A method according to claim 6 wherein the biomarker is measured by a quantitative method selected from the group consisting of an immunological or biochemical assay specific for the biomarker, an enzyme-linked immunosorbent assay (ELISA), a Western blot assay, a Northern blot assay, and a Southern blot assay.

11. A method for monitoring progression of a neurological disease in a subject comprising
(a) obtaining a cerebrospinal fluid (CSF) sample from a subject;
(b) providing a cerebrospinal fluid (CSF) correction factor for CSF obtained from a subject for at least one biomarker; and
(c) determining whether the biomarker is present in elevated amounts or concentrations in the cerebrospinal fluid (CSF) sample.

12. A method according to claim 11 wherein the neurological disease is selected from the group consisting of impaired cognition and dementia.

13. A method according to claim 11 wherein the neurological disease is selected from the group consisting of Alzheimer's Disease (AD) and Mild Cognitive Impairment (MCI).

14. A method according to claim 11 wherein the biomarker is selected from the group consisting of a tau protein and an amyloid beta (Aβ).

15. A method according to claim 11 wherein the biomarker is measured by a quantitative method selected from the group consisting of an immunological or biochemical assay specific for the biomarker, an enzyme-linked immunosorbent assay (ELISA), a Western blot assay, a Northern blot assay, and a Southern blot assay.

16. A method for assessing efficacy of a therapy for a neurological disease in a subject comprising
(a) obtaining a cerebrospinal fluid (CSF) sample from a subject;
(b) providing a cerebrospinal fluid (CSF) correction factor for CSF obtained from a subject for at least one biomarker; and
(c) determining whether the biomarker is present in elevated amounts or concentrations in the cerebrospinal fluid (CSF) sample.

17. A method according to claim 16 wherein the neurological disease is selected from the group consisting of impaired cognition and dementia.

18. A method according to claim 16 wherein the neurological disease is selected from the group consisting of Alzheimer's Disease (AD) and Mild Cognitive Impairment (MCI).

19. A method according to claim 16 wherein the biomarker is selected from the group consisting of a tau protein and an amyloid beta (Aβ).

20. A method according to claim 16 wherein the biomarker is measured by a quantitative method selected from the group consisting of an immunological or biochemical assay specific for the biomarker, an enzyme-linked immunosorbent assay (ELISA), a Western blot assay, a Northern blot assay, and a Southern blot assay.

21. A method for identifying a subject suffering from a neurological disease that may be successfully treated by an agent that affects levels of a biomarker comprising
(a) obtaining a cerebrospinal fluid (CSF) sample from a subject;
(b) providing a cerebrospinal fluid (CSF) correction factor for CSF obtained from a subject for at least one biomarker; and
(c) determining whether the biomarker is present in elevated amounts or concentrations in the cerebrospinal fluid (CSF) sample.

22. A method according to claim 21 wherein the neurological disease is selected from the group consisting of impaired cognition and dementia.

23. A method according to claim 21 wherein the neurological disease is selected from the group consisting of Alzheimer's Disease (AD) and Mild Cognitive Impairment (MCI).

24. A method according to claim 21 wherein the biomarker is selected from the group consisting of a tau protein and an amyloid beta (Aβ).

25. A method according to claim 21 wherein the biomarker is measured by a quantitative method selected from the group consisting of an immunological or biochemical assay specific for the biomarker, an enzyme-linked immunosorbent assay (ELISA), a Western blot assay, a Northern blot assay, and a Southern blot assay.


SEQUENCE LISTING (5 sequences)

SEQ ID NO: 1 - DNA, 3416 bp, Homo sapiens
gctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaa

SEQ ID NO: 2 - DNA, 120 bp, Homo sapiens
gatgcagaattccgacatgactcaggatatgaagttcatcatcaaaaattggtgttctttgcagaagatgtgggttcaaacaaaggtgcaatcattggactcatggtgggcggtgttgtc

SEQ ID NO: 3 - protein, 40 aa, Homo sapiens
DAEFRHDSGYEVHHQKLVFFAEDVGSNKGAIIGLMVGGVV

SEQ ID NO: 4 - DNA, 126 bp, Homo sapiens
gatgcagaattccgacatgactcaggatatgaagttcatcatcaaaaattggtgttctttgcagaagatgtgggttcaaacaaaggtgcaatcattggactcatggtgggcggtgttgtcatagcg

SEQ ID NO: 5 - protein, 42 aa, Homo sapiens [sequence omitted]